(19) United States
(12) Patent Application Publication
(10) Pub. No.: US 2013/0303576 A1
(43) Pub. Date: Nov. 14, 2013

(54) ENZYME INHIBITORS

(71) Applicants: Alastair David Graham Donald, Oxfordshire (GB); David Festus Charles Moffat, Abingdon (GB); Andrew James Belfield, Abingdon (GB); Carl Leslie North, Abingdon (GB); Stewart Andrew Wayne Jones, Abindgon (GB)

(72) Inventors: Alastair David Graham Donald, Oxfordshire (GB); David Festus Charles Moffat, Abingdon (GB); Andrew James Belfield, Abingdon (GB); Carl Leslie North, Abingdon (GB); Stewart Andrew Wayne Jones, Abindgon (GB)

(21) Appl. No.: 13/946,488

(22) Filed: Jul. 19, 2013

Related U.S. Application Data
(62) Division of application No. 13/202,105, filed on Oct. 13, 2011.

(30) Foreign Application Priority Data
Feb. 27, 2009 (GB) 0903480.2

(51) Int. Cl. C07D 213/58

(52) U.S. Cl. CPC C07D 213/58
USPC 514/357; 546/335

(57) ABSTRACT

Compounds of formula (I), inhibit HDAC activity: wherein A, B and D independently represent ═CH— or ═N—; W is —CH═CH—Or —CH₂CH₂—; R₁ is a carboxylic acid group (—COOH), or an ester group which is hydrolysable by one or more intracellular carboxylesterase enzymes to a carboxylic acid group; R2 and R3 are selected from the side chains of a natural or non-natural alpha amino acid, provided that neither R₂ nor R₃ is hydrogen, or R₂ and R₃, taken together with the carbon to which they are attached, form a 3-6 membered saturated cycloalkyl or heterocyclyl ring; Y is a bond, —C(═O)—, —S(═O)₂—, —C(═O)O—, —C(═O)NR′—, —C(═S)—NR′, —C(═NH)NR′ or —S(═O)₂NR— wherein R′ is hydrogen or optionally substituted C₁-C₆ alkyl; L¹ is a divalent radical of formula -(Alk¹)ₘ(Q)ₙ(Alk²)ₚ- wherein m, n, p, Q. Alk¹ and Alk² are as defined in the claims; X¹ represents a bond; —C(═O); or —S(═O)₂—; —NR₄C(═O)—, —C(═O)NR₄—, —NR₄C(═O)NR₅—, —NR₄S(═O)₂—, or —S(═O)₂NR₄— wherein R₄ and R₅ are independently hydrogen or optionally substituted C₁-C₆ alkyl; and z is 0 or 1.

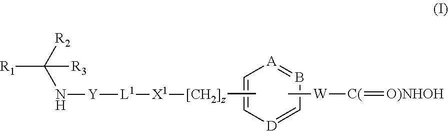

This invention relates to compounds which inhibit members of the histone deacetylase family of enzymes and to their use in the treatment of cell proliferative diseases, including cancers, polyglutamine diseases, for example Huntingdon disease, neurogenerative diseases, for example Alzheimer disease, autoimmune disease, for example rheumatoid arthritis diabetes, haematological disorders, inflammatory disease, cardiovascular disease, atherosclerosis, and the inflammatory sequelia of infection.

### BACKGROUND TO THE INVENTION

In eukaryotic cells DNA is packaged with histories, to form chromatin. Approximately 150 base pairs of DNA are wrapped twice around an octamer of histones (two each of histories 2A, 2B, 3 and 4) to form a nucleosome, the basic unit of chromatin. The ordered structure of chromatin needs to be modified in order to allow transcription of the associated genes. Transcriptional regulation is key to differentiation, proliferation and apoptosis, and is, therefore, tightly controlled. Control of the changes in chromatin structure (and hence of transcription) is mediated by covalent modifications to histories, most notably of the N-terminal tails. Covalent modifications (for example methylation, acetylation, phosphorylation and ubiquitination) of the side chains of amino acids are enzymatically mediated (A review of the covalent modifications of histones and their role in transcriptional regulation can be found in S. L. Berger, Oncogene, 2001, 20, 3007-3013. See M. Grunstein, Nature, 1997, 389, 349-352; A. P. Wolfe, Science, 1996, 272, 371-372; and P. A. Wade et al, Trends Biochem. Sci., 1997, 22, 128-132 for reviews of histone acetylation and transcription).

Acetylation of histones is associated with areas of chromatin that are transcriptionally active, whereas nucleosomes with low acetylation levels are, typically, transcriptionally silent. The acetylation status of histones is controlled by two enzyme classes of opposing activities; histone acetyltransferases (HATs) and histone deacetylases (HDACs). In transformed cells it is believed that inappropriate expression of HDACs results in silencing of tumour suppressor genes (For a review of the potential roles of HDACs in tumorigenesis see S. G. Gray and B. T. The, Curr. Mol. Med., 2001, 1, 401-429). Inhibitors of HDAC enzymes have been described in the literature and shown to induce transcriptional reactivation of certain genes resulting in the inhibition of cancer cell proliferation, induction of apoptosis and inhibition of tumour growth in animals (For review see W. K. Kelly el al, Expert Opin. Investig. Drugs, 2002, 11, 1695-1713). Such findings suggest that HDAC inhibitors have therapeutic potential in the treatment of proliferative diseases such as cancer (O. H. Kramer at al, Trends Endocrinol., 2001, 12, 294-300; a M. Vigushin and R. C. Coombes, Anticancer Drugs, 2002, 13, 1-13).

In addition, others have proposed that aberrant HDAC activity or histone acetylation is implicated in the following diseases and disorders; inflammatory disorders (F. Leoni et al, Proc. Soc. Natl. Acad. Sci., 2002, 99, 2995-3000), polyglutamine disease, for example Huntingdon disease (R. E. Hughes, Curr Biol, 2002, 12, R141-R143; A. McCampbell at al, Proc. Soc. Natl. Acad. Sci., 2001, 98, 15179-15184; E. Hockly et al, Proc. Soc. Natl. Acad. Sci., 2003, 100, 2041-2046), other neurodegenerative diseases, for example Alzheimer disease (B. Hempen and J. P. Brion, J. Neuropathol. Exp. Neurol., 1996, 55. 964-972), autoimmune disease and organ transplant rejection (S. Skov et al, Blood, 2003, 101, 1430-1438; N. Mishra et al, J. Clin. Invest., 2003, 111, 539-552), diabetes (A. L. Mosley and S. Ozcan, J. Biol. Chem., 2003, 278, 19660-19666) and diabetic complications, infection (including protozoal infection (S. J. Darkin-Rattray et al, Proc. Soc. Natl. Acad. Sci., 1996, 93, 13143-13147)) and haematological disorders including thalassemia (O. Witt et al, Blood, 2003, 101, 2001-2007). The observations contained in these manuscripts suggest that HDAC inhibition should have therapeutic benefit in these, and other related diseases.

Many types of HDAC inhibitor compounds have been suggested, and several such compounds are currently being evaluated clinically, for the treatment of cancers. For example, the following patent publications disclose such compounds:

| | | |
|---|---|---|
| U.S. Pat. No. 5,369,108 | WO 02/22577 | WO 03/092686 |
| WO 01/18171 | WO 03/075929 | WO 03/066579 |
| U.S. Pat. No. 4,254,220 | WO 03/076395 | WO 03/011851 |
| WO 01/70675 | WO 03/076400 | WO 04/013130 |
| WO 01/38322 | WO 03/076401 | WO 04/110989 |
| WO 02/30879 | WO 03/076421 | WO 04/092115 |
| WO 02/26703 | WO 03/076430 | WO 04/224991 |
| WO 02/069947 | WO 03/076422 | WO 04/076386 |
| WO 02/26696 | WO 03/082288 | WO 05/014588 |
| WO 03/082288 | WO 03/087057 | WO 05/018578 |
| WO 05/019174 | WO 05/030704 | WO 05/026907 |
| WO 05/004861 | WO 05/013958 | WO 06/016680 |
| WO 05/007091 | WO 05/028447 | |

Many of the HDAC inhibitors known in the art have a structural template, which may be represented as in formula (A):

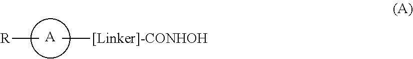

wherein ring A is a carbocyclic or heterocyclic ring system with optional substituents R, and [Linker] is a linker radical of various types. The hydroxamate group functions as a metal binding group, interacting with the metal ion at the active site of the HDAC enzyme, which lies at the base of a pocket in the folded enzyme structure. The ring or ring system A lies within or at the entrance to the pocket containing the metal ion, with the -[Linker]- radical extending deeper into that pocket linking A to the metal binding hydroxamic acid group. In the ad, and occasionally herein, the ring or ring system A is sometimes informally referred to as the “head group” of the inhibitor.

The use of prodrugs to enhance the delivery to target organs and tissues, or to overcome poor pharmacokinetic properties of the parent drug, is a well known medicinal chemistry approach. Administration of ester prodrugs, for example, which am hydrolysed by serum carboxylesterases in vivo to the active parent acids, can result in higher serum levels of the parent acid than administration of the acid itself.

### BRIEF DESCRIPTION OF THE INVENTION

We have now discovered a group of compounds which are potent and selective inhibitors of HDAC enzymes. The compounds are thus of use in medicine, for example in the treatment of disorders for which HDAC is a recognised target for therapeutic intervention. The compounds are characterised by the presence in the molecule of an α,α-disubstituted glycine motif or an α,α-disubstituted glycine ester motif which is hydrolysable by an intracellular carboxylesterase. Compounds of the invention having the lipophilic α,α-disubstituted glycine ester motif cross the cell membrane, and are hydrolysed to the acid by the intracellular carboxylesterases. The polar hydrolysis product accumulates in the cell since it does not readily cross the cell membrane. Hence the HDAC inhibitory activity of the compound is prolonged and enhanced within the cell. The compounds of the invention are related to the HDAC inhibitors encompassed by the disclosures in International Patent Application WO 2008/040934. The latter compounds have an α-monosubstituted glycine ester motif which also enables the compounds to cross the cell membrane into the cell where they are hydrolysed to the corresponding acid by intracellular carboxylesterases. However, that publication does not suggest that α,α-disubstituted glycine ester conjugates can be hydrolysed by intracellular carboxylesterases. In fact, it appears that the ability of the intracellular carboxyl esterases, principally hCE-1, hCE-2 and hCE-3, to hydrolyse α,α-disubstituted glycine esters has not previously been investigated.

The general concept of conjugating an oz-mono substituted glycine ester motif to a modulator of an intracellular, enzyme or receptor, to obtain the benefits of intracellular accumulation of the carboxylic acid hydrolysis product is disclosed in our International Patent Application WO 2006/117567. However, this publication does not suggest that α,α-disubstituted glycine ester conjugates can be hydrolysed by intracellular carboxylesterases. As mentioned above, it appears that the ability of the intracellular carboxyl esterases, principally hCE-1, hCE-2 and hCE-3, to hydrolyse α,α-disubstituted glycine esters has not previously been investigated.

This invention therefore makes available a new class of HDAC inhibitors having pharmaceutical utility in the treatment of diseases such as cancers or inflammation which benefit from intracellular inhibition of HDAC, which compounds have an α,α-disubstituted glycine ester grouping which facilitates penetration of the agent through the cell wall, and thereby allows intracellular carboxylesterase activity to hydrolyse the ester to release the parent acid. Being charged, the acid is not readily transported out of the cell, where it therefore accumulates to increase the intracellular concentration of active HDAC inhibitor. This leads to increases in potency and duration of action.

The compounds of the present invention differ from those described in copending International patent application no. WO 2008/040934 in that the amino acid ester conjugate part of the latter compounds is mono-substituted on the alpha carbon, whereas in the present compounds that alpha carbon is di-substituted. This structural difference can be beneficial, since the present α-disubstituted glycine ester conjugates tend to have lower HDAC inhibitory activity than their mono-alpha substituted counterparts, and in such cases the HDAC inhibitory activity of the present compounds is thus primarily exerted in the cells in which their hydrolysis product accumulates, rather than as a general systemic effect.

### DETAILED DESCRIPTION OF THE INVENTION

According to the invention there is provided a compound of formula (I):

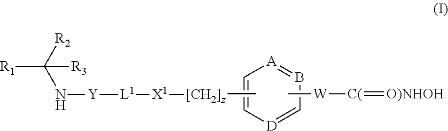

wherein

A, B and D independently represent ═CH— or ═N—;

W is a divalent radical —CH═CH— or CH₂CH₂—:

R₁ is a carboxylic acid group (—COOH), or an ester group which is hydrolysable by one or more intracellular carboxylesterase enzymes to a carboxylic acid group;

R₂ and R₃ are selected from the side chains of a natural or non-natural alpha amino acid, provided that neither R₂ nor R₃ is hydrogen, or R₂ and R₃, taken together with the carbon to which they are attached, may form a 3-6 membered saturated spiro cycloalkyl or heterocyclyl ring.

Y is a bond, —C(═O)—, —S(═O)₂—, —C(═O)O—, —C(═O)NR—, —C(═S)—NR′, —C(═NH)NR′ or —S(═O)₂NR′— wherein R′ is hydrogen or optionally substituted C₁-C₆ alkyl;

L¹ is a divalent radical of formula -(Alk¹)ₘ(Q)ₘ(Alk²)ₚ— wherein
m, n and p are independently 0 or 1,
Q is (i) an optionally substituted divalent mono- or bicyclic carbocyclic or heterocyclic radical having 5-13 ring members, or (ii), in the case where both m and p are 0, a divalent radical of formula -X²-Q¹- or -Q¹-X²- wherein X² is —O—, S— or NR^{A}— wherein R^{A} is hydrogen or optionally substituted C₁-C₃ alkyl, and Q¹ is an optionally substituted divalent mono- or bicyclic carbocyclic or heterocyclic radical having 5-13 ring members,
Alk¹ and Alk² independently represent optionally substituted divalent C₃-C₇ cycloalkyl radicals, or optionally substituted straight or branched, C₁-C₆ alkylene, C₂-C₆ alkenylene, or C₂-C₆ alkynylene radicals which may optionally contain or terminate in an ether (—O—), thioether (—S—) or amino (—NR^{A}—) link wherein R^{A} is hydrogen or optionally substituted C₁-C₃ alkyl;

X¹ represents a bond; —C(═O); or —S(═O)₂—; —NR₄C(═O)—, —C(═O)NR₄—, —NR₄C(═O)NR₅—, —NR₄S(═O)₂—, —S(═O)₂NR₄— wherein R₄ and R₅ are independently hydrogen or optionally substituted C₁-C₆ alkyl; and

z is 0 or 1.

Compounds of formula (I) above may be prepared in the form of salts, especially pharmaceutically acceptable salts, N-oxides, hydrates, solvates and polymorphic forms thereof. Any claim to a compound herein, or reference herein to “compounds of the invention”, “compounds with which the invention is concerned”, “compounds of formula (I)” and the like, includes salts, N-oxides, hydrates, solvates and polymorphs of such compounds. Reference herein to “ester compounds of the invention”, “ester compounds with which the invention is concerned”, “ester compounds of formula (I)” and the like, refers to compounds of formula (I) in which R₁ is an ester group which is hydrolysable by one or more intracellular carboxylesterase enzymes to a carboxylic acid group, and includes salts, N-oxides, hydrates, solvates and polymorphs of such compounds.

Although the above definition potentially includes molecules of high molecular weight, it is preferable, in line with general principles of medicinal chemistry practice, that the compounds with which this invention is concerned should have molecular weights of no more than 600.

The ester compounds of the invention are hydrolysed by intracellular carboxylesterases after penetrating the cell wall, and are thus converted to the corresponding carboxylic acids. The latter form part of the invention because they are active HDAC inhibitors when released in the cell, but they are not generally useful as drugs for administration per se to a subject. It is the ester compounds of the invention which are considered useful for administration.

Therefore, in another broad aspect the invention provides the use of an ester compound of the invention in the preparation of a composition for inhibiting the activity of histone deacetylase.

The ester compounds with which the invention is concerned may be used for the inhibition of histone deacetylase activity, ex vivo or in viva

In one aspect of the invention, the ester compounds of the invention may be used in the preparation of a composition for the treatment of cell-proliferation disease, for example cancer cell proliferation and autoimmune diseases.

In another aspect, the invention provides a method for the treatment of the foregoing disease types, which comprises administering to a subject suffering such disease an effective amount of an ester compound of the invention.

### Terminology

The term “ester” or “esterfied carboxyl group” means a group R₉O(C═O)— in which R₉ is the group characterising the ester, notionally derived from the alcohol R₉OH.

As used herein, the term “(C₈-C₉)alkyl” wherein a and b are integers refers to a straight or branched chain alkyl radical having from a to b carbon atoms. Thus when a is 1 and b is 6, tor example, the term includes methyl, ethyl, n-propyl, isopropyl, n-butyl, isobutyl, sec-butyl, t-butyl, n-pentyl and n-hexyl.

As used herein the term “divalent (Cₐ-C_{b})alkylene radical” wherein a and b are integers refers to a saturated hydrocarbon chain having from a to b carbon atoms and two unsatisfied valences.

As used herein the term “(Cₐ-C_{b})alkenyl” wherein a and b are integers refers to a straight or branched chain alkenyl moiety having from a to b carbon atoms having at least one double bond of either E or Z stereochemistry where applicable. The term includes, for example, vinyl, allyl, 1- and 2-butenyl and 2-methyl-2-propenyl.

As used herein the term “divalent (Cₐ-C_{b})alkenylene radical” means a hydrocarbon chain having from a to b carbon atoms, at least one double bond, and two unsatisfied valences.

As used herein the term “Cₐ-C_{b} alkynyl” wherein a and b are integers refers to straight chain or branched chain hydrocarbon groups having from a to b carbon atoms and having in addition one triple bond. This term would include for example, ethynyl, 1-propynyl, 1- and 2-butynyl, 2-methyl-2-propynyl, 2-pentynyl, 3-pentynyl, 4-pentynyl, 2-hexynyl, 3-hexynyl, 4-hexynyl and 5-hexynyl.

As used herein the term “divalent (Cₐ-C_{b})alkynylene radical” wherein a and b are integers refers to a divalent hydrocarbon chain having from a to b carbon atoms, and at least one triple bond.

As used herein the term “carbocyclic” refers to a mono-, bi- or tricyclic radical having up to 16 ring atoms, all of which are carbon, and includes aryl and cycloalkyl.

As used herein the term “cycloalkyl” refers to a monocyclic saturated carbocyclic radical having from 3-8 carbon atoms and includes, for example, cyclopropyl, cyclobutyl, cyclopentyl, cyclohexyl, cycloheptyl and cyclooctyl.

As used herein the unqualified term “aryl” refers to a mono-, bi- or hi-cyclic carbocyclic aromatic radical, and includes radicals having two monocyclic carbocyclic aromatic rings which are directly linked by a covalent bond. Illustrative of such radicals are phenyl, biphenyl and naphthyl.

As used herein the unqualified term “heteroaryl” refers to a mono-, bi- or tri-cyclic aromatic radical containing from 1 to 4 heteroatoms selected from S, N and O, and includes radicals having two such monocyclic rings, or one such monocyclic ring and one monocyciic aryl ring, which are directly linked by a covalent bond. Illustrative of such radicals are thienyl, benzthienyi, furyi, benzfuryl, pyrrolyl, imidazolyi, henzimidazolyl, thiazolyl, benzthiazolyl, isothiazolyl, benzisothiazolyl, pyrazolyl, oxazoiyi, benzoxazolyl, isoxazolyl, benzisoxazolyl, isothiazolyl, triazolyl, benztriazolyl, thiadiazolyl, oxadiazolyl, pyridinyl, pyridazinyl, pyrazinyl, triazinyl, indolyl and indazolyl.

As used herein the unqualified term “heterocyclyl” or “heterocyclic” includes “heteroaryl” as defined above, and in its non-aromatic meaning relates to a mono-, bi- or tri-cyclic non-aromatic radical containing from 1 to 4 heteroatoms selected from S, N and O, and to groups consisting of a monocyclic non-aromatic radical containing one or more such heteroatoms which is covalently linked to another such radical or to a monocyclic carbocyclic radical. Illustrative of such radicals are pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, morpholinyl, benzfuranyl, pyranyl, isoxazolyl, benzimidazolyl, methylenedioxyphenyl, ethylenedioxyphenyl, maleimido and succinimido groups.

Unless otherwise specified in the context in which it occurs, the term “substituted” as applied to any moiety herein means substituted with up to four compatible substituents, each of which independently may be, for example, (C₁-C₆)alkyl, (C₁-C₆)alkoxy, hydroxy, hydroxy(C₁-C₆)alkyl, mercapto, mercapto(C₁-C₆)alkyl, (C₁-C₆)alkylthio, phenyl, halo (including fluoro, bromo and chloro), trifluoromethyl, trifluoromethoxy, nitro, nitrile (—CN), oxo, —COOH, —COOR^{A}, —COR^{A}, —SO₂R^{A}, —CONH₂, —SO₂NH₂, —CONHR^{A}, —SO₂NHR^{A}, —CONR^{A}R^{B}, —SO₂NR^{A}R^{B}, —NH₂, —NHR^{A}, —NR^{A}R^{B}, —OCONH₂, —OCONHR^{A}, —OCONR^{A}R^{B}, —NHCOR^{A}, —NHCOOR^{A}, —NR⁵COOR^{A}, —NHSO₂OR^{A}, —NR⁵SO₂OH, —NR⁵SO₂OR^{A}, —NHCONH₂, —NR^{A}CONH₂, —NHCONHR^{B}, —NR^{A}CONHR^{B}, —NHCONR^{A}R⁵, or —NR^{A}CONR^{A}R⁵ wherein R^{A} and R⁵ are independently a (C₁-C₆)alkyl, (C₃-C₆) cycloalkyl, phenyl or monocyclic heteroaryl having 5 or 6 ring atoms, or R^{A} and R^{B} when attached to the same nitrogen atom form a cyclic amino group (for example morpholino, piperidinyl, piperazinyl, or tetrahydropyrrolyl). An “optional substituent” may be one of the foregoing substituent groups.

As used herein, the term “nitrogen substituent” means a substituent on a nitrogen atom which is selected from the following:
amino (C₁-C₆)alkyl e.g. aminoethyl, (C₁-C₃)alkylamino-(C₁-C₆)alkyl-, (C₁-C₃)dialkylamino-(C₁-C₆)alkyl, hydroxy(C₁-C₆)alkyl e.g. hydroxyethyl, (C₁-C₃)alkoxy-(C₁-C₆)alkyl- e.g. methoxyethyl, mercapto(C₁-C₃)alkyl, (C₁-C₃)alkylmercapto-(C₁-C₆)alkyl-, carboxamido(C₁-C₆)alkyl e.g. —CH₂CONH₂, aminosulphonyl(C₁-C₆)alkyl- e.g. —CH₂SO₂NH₂, (C₁-C₃)alkylaminosulphonyl- (C₁-C₆)alkyl- e.g. —CH₂SO₂NHMe, (C₁-C₃)dialkylaminosulphonyl-(C₁-C₆)alkyl e.g. —CH₂SO₂NMe₂, (C₁-C₆)alkanoyl, (C₁-C₆)alkylsulphonyl, aminosulphonyl (—SO₂NH₂), (C₁-C₆)alkylaminosulphonyl e.g. —SO₂NHMe, (C₁-C₆)dialkylaminosulphonyl e.g. —SO₂NMe₂, optionally substituted phenylaminosulphonyl, carboxamido (—CONH₂), (C₁-C₆)alkylaminocarbonyl, (C₁-C₆)dialkylaminocarbonyl, morpholinyl(C₁-C₆)alkyl, imidazolyl(C₁-C₆)alkyl, triazolyl(C₁-C₆)alkyl, or monocyclic heterocycloalkyl(C₁-C₆)alkyl, optionally substituted in the imidazolyl, triazolyl or heterocyclyl ring, e.g. piperidinyl(C₁-C₆)alkyl, piperazinyl(C₁-C₆)alkyl or 4-((C₁-C₆)alkyl)piperazinyl(C₁-C₆)alkyl.

The term “side chain of a natural or non-natural alpha-amino acid” refers to the group R¹ in a natural or non-natural amino acid of formula NH₂—CH(R¹)—COOH.

Examples of side chains of natural alpha amino acids include those of alanine, arginine, asparagine, aspartic acid, cysteine, cystine, glutamic acid, histidine, 5-hydroxylysine, 4-hydroxyproline, isoleucine, leucine, lysine, methionine, phenylalanine, proline, swine, threonine, tryptophan, tyrosine, valine, α-aminoadipic acid, α-amino-n-butyric acid, 3,4-dihydroxyphenylalanine, homoserine, β-methylserine, ornithine, pipecolic acid, and thyroxine.

Natural alpha-amino acids which contain functional substituents, for example amino, carboxyl, hydroxy, mercapto, guanidyl, imidazolyl, or indolyl groups in their characteristic side chains include arginine, lysine, glutamic acid, aspartic acid, tryptophan, histidine, serine, threonine, tyrosine, and cysteine. When R₂ in the compounds of the invention is one of those side chains, the functional substituent may optionally be protected.

The term “protected” when used in relation to a functional substituent in a side chain of a natural alpha-amino acid means a derivative of such a substituent which is substantially non-functional. For example, carboxyl groups may be esterified (for example as a C₁-C₆ alkyl ester), amino groups may be converted to amides (for example as a NHCOC₁-C₆ alkyl amide) or carbamates (for example as an NHC(═O)OC₁-C₆ alkyl or NHC(═O)OCH₂Ph carbamate), hydroxyl groups may be converted to ethers (for example an OC₁-C₆ alkyl or a O(C₁-C₆ alkyl)phenyl ether) or esters (for example a OC(═O)C₁-C₆ alkyl ester) and thiol groups may be converted to thioethers (for example a tert-butyl or benzyl thioether) or thioesters (for example a SC(═O)C₁-C₆ alkyl thioester).

Examples of side chains of non-natural alpha amino acids include those referred to below in the discussion of suitable R₂ and R₃ groups for use in compounds of the present invention.

Compounds of the invention may exist in one or more geometrical, optical, enantiomeric, diastereomeric and tautomeric forms, including but not limited to cis- and trans-forms, E- and Z-forms, R-, S- and meso-forms, keto-, and enol-forms. Unless otherwise stated a reference to a particular compound includes all such isomeric forms, including racemic and other mixtures thereof. Where appropriate such isomers can be separated from their mixtures by the application or adaptation of known methods (e.g. chromatographic techniques and recrystallisation techniques). Where appropriate such isomers may be prepared by the application of adaptation of known methods (e.g. asymmetric synthesis).

As used herein the term “salt” includes base addition, acid addition and ammonium salts. As briefly mentioned above compounds of the invention which are acidic can form salts, including pharmaceutically acceptable salts, with bases such as alkali metal hydroxides, e.g. sodium and potassium hydroxides; alkaline earth metal hydroxides e.g. calcium, barium and magnesium hydroxides; with organic bases e.g. N-methyl-D-glucamine, choline tris(hydroxymethyl)amino-methane, L-arginine, L-lysine, N-ethyl piperidine, dibenzylamine and the like. Those compounds of the invention which are basic can form salts, including pharmaceutically acceptable salts with inorganic acids, e.g. with hydrohalic acids such as hydrochloric or hydrobromic acids, sulphuric acid, nitric acid or phosphoric acid and the like, and with organic acids e.g. with acetic, trifluoroacetic, tartaric, succinic, fumaric, maleic, malic, salicylic, citric, methanesulphonic, p-toluenesulphonic, benzoic, benzenesulfonic, glutamic, lactic, and mandelic acids and the like. Those compounds (I) which have a basic nitrogen can also form quaternary ammonium salts with a pharmaceutically acceptable counter-ion such as chloride, bromide, acetate, formate, p-toluenesulfonate, succinate, hemi-succinate, naphthalene-bis sulfonate, methanesulfonate, trifluoroacetate, xinafoate, and the like. For a review on salts, see Handbook of Pharmaceutical Salts: Properties, Selection, and Use by Stahl and Wermuth (Wiley-VCH, Weinheim, Germany, 2002).

It is expected that compounds of the invention may be prepared in the form of hydrates, and solvates. Any reference herein, including the claims herein, to “compounds with which the invention is concerned” or “compounds of the invention” or “the present compounds”, and the like, includes reference to salts, hydrates, and solvates of such compounds. The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Individual compounds of the invention may exist in an amorphous form and/or several polymorphic forms and may be obtained in different crystal habits. Any reference herein, including the claims herein, to “compounds with which the invention is concerned” or ‘compounds of the invention’ or “the present compounds”, and the like, includes reference to the compounds irrespective of amorphous or polymorphic form.

Some compounds of the invention, having a nitrogen atom in an aromatic ring, may form N-oxides, and the invention includes compounds of the invention in their N-oxide form.

As stated above, the esters of the invention are primarily prodrugs of the corresponding carboxylic acids to which they are converted by intracellular esterases. However, for so long as they remain unhydrolysed, the esters may have HDAC inhibitory activity in their own right. The compounds of the invention include not only the ester, but also the corresponding carboxylic acid hydrolysis products, but it is the esters which are intended for administration to patients.

In the compounds of the invention, in any compatible combination, and bearing in mind that the compounds preferably have a molecular weight of less than 600:

The hydroxamate Group —C(═O)NHOH
In the compounds of the invention, the hydroxamate group functions as a metal binding group, interacting with the metal ion at the active site of the HDAC enzyme, which lies at the base of a pocket in the folded enzyme structure.

### The Ring Containing A, B and

Each of A, B and D may be —CH═, or at least one of A, B and D may be —N═. For example, A may be —CH═ and B and may each be —N═. In many embodiments each of A, B and D is —CH═, or each of A and is —CH═ while B is —N═.
   The Radical —Y-L¹-X¹—[CH₂]_{z}—
L¹ may be selected from:
   (i) a bond;
   (ii) —O—, —S—, —C(═O)—, —S(═O)₂—, —NR¹⁰, —C(═O)NR¹⁰—, —S(═O)₂NR¹⁰—, —NR¹⁰C(═O)—, —NR¹⁰S(═O)₂—, —NR¹⁰(CH₂)ₘ—, —NR¹⁰C(═O)(CH₂)ₘ—, —NR¹⁰S(═O)₂(CH₂)ₘ, NR²⁰C(═O)NR¹⁰—, —NR¹⁰C(═O)(CH₂)ₘAr-, or —NR¹⁰S(═O)₂(CH₂)ₘAr- wherein R¹⁰ and R²⁰ are independently hydrogen. C₁-C₄ alkyl, or a nitrogen substituent, m is 0, 1, 2 or 3, and Ar is a divalent phenyl radical or a divalent mono-, or bi-cyclic heteroaryl radical having 5 to 13 ring members; and
   (iii) an optionally substituted, straight or branched, C₁-C₆ alkylene, C₂-C₆ alkenylene or C₂-C₆ alkynylene radical which may optionally contain or terminate in an ether (—O—), thioether (—S—) or amino (—NR^{A}—) link wherein R^{A} is hydrogen, C₁-C₃ alkyl, or a nitrogen substituent;
In the radical L¹, Alk¹ and Alk², when present, may be selected from, for example, —CH₂—, —CH₂CH₂—, —CH₂CH₂CH₂—, CH₂O—, CH₂CH₂O—, CH₂CH₂CH₂)—, and divalent cyclopropyl, cyclopentyl and cyclohexyl radicals.
Also in the radical L¹, Q¹ may be, for example, 1,4-phenylene.
Also in the radical L¹, m and p may both be 0, or n and p may be 0 while m is 1, or m, n and p may all be 0.
X¹ may be, for example, —NR₃—, —S—, —O—, —C(═O)NR₃—, —NR₃C(═O)—, or —C(═O)O—, wherein R₃ is hydrogen, C₁-C₆ alkyl, or a nitrogen substituent, or in other cases a bond.
In the radical L¹, Alk¹ and Alk², when present, may be selected from —CH₂—, —CH₂CH₂—, —CH₂CH₂CH₂—, and divalent cyclopropyl, cyclopentyl and cyclohexyl radicals.
In the radical L¹, Q¹ may be, for example, a divalent phenyl radical or a mono-, or bicyclic heteroaryl radical having 5 to 13 ring members, such as 1,4-phenylene.
Specific examples of the radical -L¹-X¹—[CH₂]_{z}— are —(CH₂)₃NH—, —CH₂C(═O)NH—, —CH₂CH₂C(═O)NH—, —CH₂C(O)O—, —CH₂S—, —CH₂CH₂C(O)O—, —(CH₂)₄NH—, —CH₂CH₂S—, —CH₂O, —CH₂CH₂O—

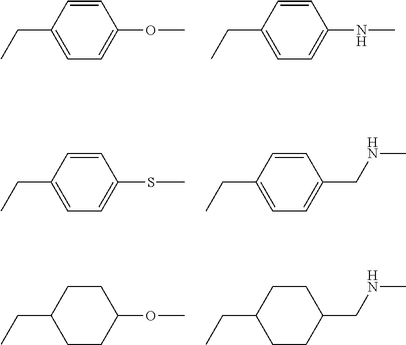

Specific examples of the radical —Y-L¹-X¹—[CH₂]_{z}— are C(═O)—, —C(O)NH—, —(CH₂)ᵥ—, —(CH₂)ᵥO—, —C(═O)—(CH₂)ᵥ—, —C(═O)(CH₂)ᵥO—, —C(═O)NH(CH₂)_{w}—, —C(═O)NH(CH₂)_{w}O—

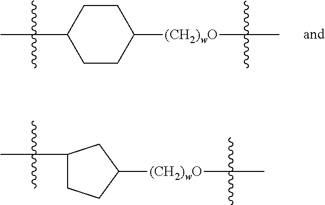

wherein v is 1, 2, 3 or 4 and w is 1, 2 or 3.

Amongst preferred Y-L¹-X¹—[CH₂]_{z}— radicals are —CH₂—, and —CH₂O—, most preferably —CH₂—.

The group R₁

Compounds of the invention wherein R₁ is a carboxylic acid group are the intracellular hydrolysis products of the corresponding esters of the invention. Although such carboxylic acids have HDAC inhibitory activity, it is preferred that they be generated in the cell by the action of an intracellular esterase after administration of the corresponding compound in which R₁ is an ester group.

The ester group R₁ must be one which in the compound of the invention is hydrolysable by one or more intracellular carboxylesterase enzymes to a carboxylic acid group. Intracellular carboxylesterase enzymes capable of hydrolysing the ester group of a compound of the invention to the corresponding acid include the three known human enzyme isotypes hCE-1, hCE-2 and hCE-3. Although these are considered to be the main enzymes, other enzymes such as biphenylhydrolase (BPH) may also have a role in hydrolysing the ester. In general, if the canboxylesterase hydrolyses the free amino acid ester to the parent acid it will also hydrolyse the ester motif when covalently conjugated to the inhibitor. Hence, the broken cell assay and/or the isolated carboxylesterase assay described herein provide a straightforward, quick and simple first screen for esters which have the required hydrolysis profile. Ester motifs selected in that way may then be re-assayed in the same carboxylesterase assay when conjugated to the inhibitor via the chosen conjugation chemistry, to confirm that it is still a carboxylesterase substrate in that background. Esters which are hydrolysable by intracellular carboxylesterases include those ester groups present in compounds prepared in International patent applications WO 2006/117587, WO 2006/117549, WO 2006/117548, WO 2006/117570, WO 2006/1/ 7552, WO 2007/129036, WO 2007/129020, WO 2007/132146, WO 2007/129040, WO 2007/129048, WO 2007/129005, WO 2008/040934, WO 2008/050096, WO 2008/050078, WO 2008/53131, WO 2008/053157, WO 2008/053185, WO 2008/053182, WO 2008/053158, WO 2008/053136, WO 2009/060160, WO2009/106848, WO 2009/106844, and WO 2009/130453.

Subject to the requirement that they be hydrolysable by intracellular carboxylesterase enzymes, examples of particular ester groups R₁ include those of formula —(C═O)OR₁₂ wherein R₁₂ is R₇R₈CR₉— wherein
(i) R₇ is hydrogen or optionally substituted (C₁-C₃)alkyl-(Z¹)ₐ-[(C₁-C₃)alkyl]_{b}- or (C₂-C₃)alkenyl-(Z¹)ₐ-[(C₁-C₃)alkyl]_{b}- wherein a and b are independently 0 or 1 and Z¹ is —O—, —S—, NR₁₃— wherein R₁₃ is hydrogen or (C₁-C₃)alkyl; and R₈ and R₉ are independently hydrogen or (C₁-C₃)alkyl-;
(ii) R₇ is hydrogen or optionally substituted R₁₄R₁₅N—(C₁-C₃)alkyl- wherein R₁₄ is hydrogen or (C₁-C₃)alkyl and R₁₅ is hydrogen or (C₁-C₃)alkyl; or R₁₄ and R₁₅ together with the nitrogen to which they are attached form an optionally substituted monocyclic heterocyclic ring of 5- or 6- ring atoms or bicyclic heterocyclic ring system of 8 to 10 ring atoms, and R₈ and R₉ are independently hydrogen or (C₁-C₃)alkyl-; or
(iii) R₇ and R₉ taken together with the carbon to which they are attached form an optionally substituted monocyclic carbocyclic ring of from 3 to 7 ring atoms or bicyclic carbocyclic ring system of 8 to 10 ring atoms, or bridged monacyclic carbocyclic ring system of 7 to 10 ring atoms, and Rₐ is hydrogen.

In cases (i), (ii) and (iii) above, “alkyl” includes fluoroalkyl.

Within these classes (i), (ii) and (iii), R_{g} is often hydrogen. Specific examples of R₁₂ include methyl, trifluoromethyl, ethyl, n- or iso-propyl, n-, sec- or tert-buty, cyclopentyl, methyl-substituted cyclopentyl, cyclohexyl, allyl, bicyclo[2.2.1]hept-2-yl, 2,3-dihydro-1H-inden-2-yl, phenyl, benzyl, 2-, 3- or 4-pyridylmethyl, N-methylpiperidin-4-yl, tetrahydrofuran-3-yl or methoxyethyl. Currently preferred is where R₁₂ is cyclopentyl.

Macrophages are known to play a key role in inflammatory disorders through the release of cytokines in particular TNFα and IL-1 (van Roon et al, Arthritis and Rheumatism, 2003, 1229-1238). In rheumatoid arthritis they are major contributors to the maintenance of joint inflammation and joint destruction. Macrophages are also involved in tumour growth and development (Naldini and Carrara, Curr Drug Targets Inflamm Allergy, 2005, 3-8). Hence agents that selectively target macrophage cell proliferation could be of value in the treatment of cancer and autoimmune disease. Targeting specific cell types would be expected to lead to reduced side-effects. It has been found that macrophages contain the human carboxylesterase hCE-1 whereas other cell types do not. In the general formula (I) when the nitrogen of the esterase motif R₁C(R₂)(R₃)NH— is not directly linked to a carbonyl (—C(═O)—), i.e. when Y is not a —C(═O), —C(═O)O— or —C(═O)NR₃— radical, the ester will only be hydrolysed by hCE-1 and hence the inhibitors will only accumulate in macrophages. Herein, unless “monocyte” or “monocytes” is specified, the term macrophage or macrophages will be used to denote macrophages (including tumour associated macrophages) and/or monocytes.

Substituents R₂ and R₃

The substituents R₂ and R₃ may be regarded as the α-substituents of an α,α-disubstituted glycine or an α,α-disubstituted glycine ester. These substituents may therefore be selected from the side chains of a natural or non-natural alpha-amino acid other than glycine, and in such side chains any functional groups may be protected.

Examples of the side chains of natural and non natural alpha- amino acids other than glycine include those of the alpha amino acids conjugated to various enzyme inhibitors in compounds prepared in International patent applications WO 2006/117567, WO 2006/117549, WO 2006/117548, WO 2006/117570, WO 2006/117552, WO 2007/129036, WO 2007/129020, WO 2007/132146, WO 2007/129040, WO 2007/129048, WO 2007/129005, WO 2008/040934, WO 2008/050096, WO 2008/050078, WO 2008/53131, WO 2008/053157, WO 2008/053185, WO 2008/053182, WO 2008/053158, WO 2008/053136, WO 2009/060160, WO2009/106848, WO 2009/106844, and WO 2009/130453.

Examples of R₂ and R₃ include phenyl, and groups of formula —CRₐR_{b}R_{c} in which:
each of Rₐ, R_{b} and R_{c} is independently hydrogen, (C₁-C₆)alkyl, (C₂-C₆)alkenyl, (C₂-C₆)alkynyl, phenyl(C₁-C₆)alkyl, (C₃-C₈)cycloalkyl; or
R_{c} is hydrogen and Rₐ and R_{b} are independently phenyl or heteroaryl such as pyridyl; or
R_{c} is hydrogen, (C₁-C₆)alkyl, (C₂-C₆)alkenyl, (C₂-C₆)alkynyl, phenyl(C₁-C₆)alkyl, or (C₃-C₆)cycloalkyl, and Rₐ and R_{b} together with the carbon atom to which they are attached form a 3 to 8 membered cycloalkyl or a 5- to 6-membered heterocyclic ring; or
Rₐ, R_{b} and R_{c} together with the carbon atom to which they are attached form a bicyclic ring (for example adamantyl);
Rₐ and R_{b} are each independently (C₁-C₆)alkyl, (C₂-C₆)alkenyl, (C₂-C₆)alkynyl, phenyl(C₁-C₆)alkyl, or a group as defined for R_{c} below other than hydrogen, or Rₐ and R_{b} together with the carbon atom to which they are attached form a cycloalkyl or heterocyclic ring, and R_{c} is hydrogen, —OH, —SH, halogen, —CN, —CO₂H, (C₁-C₄ perfluoroalkyl, —CH₂OH, —O(C₁-C₆)alkyl, —O(C₂-C₆)alkenyl, —S(C₁-C₆)alkyl, —SO(C₁-C₆)alkyl, —SO₂(C₁-C₆) alkyl, —S(C₂-C₆)alkenyl, —SO(C₂-C₆)alkenyl, —SO₂(C₂-C₆)alkenyl or a group -Q-W wherein Q represents a bond or —O—, —S—, —SO— or —SO₂— and W represents a phenyl, phenylalkyl, (C₃-C₈)cycloalkyl, (C₃-C₈)cycloalkylalkyl, (C₄-C₈)cycloalkenyl, (C₄-C₈)cycloalkenylalkyl, heteroaryl or heteroarylalkyl group, which group W may optionally be substituted by one or more substituents independently selected from, hydroxyl, halogen, —CN, —CONH₂, —CONH(C₁-C₆)alkyl, —CONH(C₁-C₆alkyl)₂, —CHO, —CH₂OH, (C₁-C₄)perfluoroalkyl, —O(C₁-C₆)alkyl, —S(C₁-C₆)alkyl, —SO(C₁-C₆)alkyl, —SO₂(C₁-C₆)alkyl, —NO₂, —NH₂, —NH(C₁-C₆)alkyl, —N((C₁-C₆)alkyl)₂, —NHCO(C₁-C₆)alkyl, (C₁-C₆)alkyl, (C₂-C₆)alkenyl, (C₂-C₆)alkynyl, (C₃-C₈)cycloalkyl, (C₄-C₈)cycloalkenyl, phenyl or benzyl.

Alternatively, the substituents R₂ and R₃, taken together with the carbon to which they are attached, may form a 3-6 membered saturated spiro cycloalkyl ring, such as a cyclopropyl, cyclobutyl, cyclopentyl or cyclohexyl ring or spiro heterocyclyl ring such as a piperidin-4-yl ring.

In some cases, at least one of the substitutents R₂ and R₃ is a C₁-C₆ alkyl substituent, for example methyl, ethyl, or n- or iso-propyl.

In some embodiments, one of the substitutents R₂ and R₃ is a C₁-C₆ alkyl substituent, for example methyl, ethyl, or n- or iso-propyl, and the other is selected from the group consisting of methyl, ethyl, n- and iso-propyl, n-, sec- and tert-butyl, phenyl, benzyl, thienyl, cyclohexyl, and cyclohexylmethyl.

In particular cases, one of the substitutents R₂ and R₃ is methyl, and the other is methyl or benzyl. In other particular cases, R₂ and R₃, taken together with the carbon to which they are attached, form a cyclopropyl, cyclobutyl, cyclopentyl or cyclohexyl ring.

For compounds of the invention which are to be administered systemically, esters with a slow rate of carboxylesterase cleavage are preferred, since they are less susceptible to pre-systemic metabolism. Their ability to reach their target tissue intact is therefore increased, and the ester can be converted inside the cells of the target tissue into the acid product. However, for topical administration, where the ester is either directly applied to the target tissue or directed there by, for example, inhalation, it will often be desirable that the ester has a rapid rate of esterase cleavage, to minimise systemic exposure and consequent unwanted side effects. In the compounds of this invention, if the carbon adjacant to the alpha carbon of the alpha amino acid ester is monosubstituted, i.e. R₂ is CH₂R^{z} (R^{z} being the mono-substituent) then the esters tend to be cleaved more rapidly than if that carbon is di- or tri-substituted, as in the case where R₂ is, for example, phenyl or cyclohexyl.

One subset of the compounds of the invention has formula (IA):

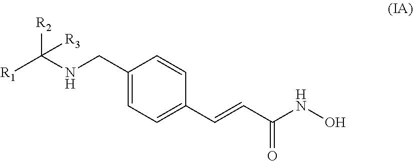

wherein R₁, R₂ and R₃ are as defined and further discussed above.

Another subset of the compounds of the invention has formula (IB):

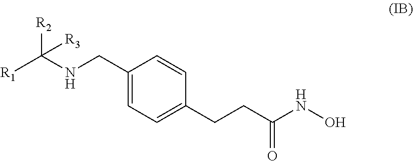

wherein R₁, R₂ and R₃ are as defined and further discussed above.

Yet another subset of the compounds of the invention has formula (IC);

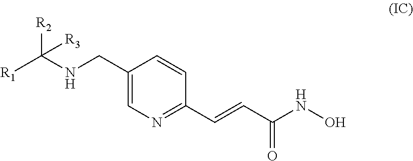

wherein R₁, R₂ and R₃ are as defined and further discussed above.

Yet another subset of the compounds of the invention has formula (ID);

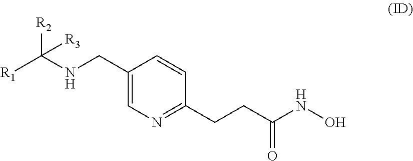

wherein R₁, R₂ and R₃ are as defined and further discussed above.

A currently preferred subset of the compounds of the invention has formula (IE)

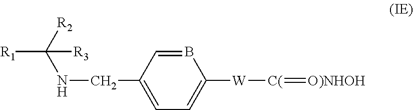

wherein R₁, W and B are as defined in claim 1, one of R₂ and R₃ is methyl, and the other is methyl, ethyl, n- or iso-propyl, benzyl or n, sec or tart butyl; or R₂ and R₃ taken together with the carbon to which they are attached form a cyclopropyl, cyclobutyl, cyclopentyl or cyclohexyl ring. In this subset, compounds wherein R₁ is an ester group of formula R₁₂C(═O)—, wherein R₁₂ is cyclopentyl, are often preferred. Also in this subclass compounds wherein W is —CH═CH— are often preferred.

In each of the subsets (1A)-(IE) above compounds wherein R₁ is an ester group as defined and discussed in relation to formula (I) are preferred as the compounds for administration to patients, since it is as esters that they enter cells and are hydrolysed intracellularly to the corresponding acids.

Specific compounds of the invention include those of the Examples, whether or not in salt form.

### Utilities

As mentioned above, the compounds with which the invention is concerned are of use for inhibition of HDAC activity. Inhibition of HDAC activity is a mechanism for treatment of a variety of diseases, including cell proliferative disease such as cancer (including malignancies of the monocytic cell lineage, e.g., juvenile myelomonocytic leukaemia) and psoriasis, polyglutamine disease such as Huntingdon's disease, neurogenerative disease such as Alzheimers disease, autoimmune disease such as rheumatoid arthritis (including systemic juvenile idiopathic arthritis), diabetes, haematological disease, inflammatory disease, cardiovascular disease, atherosclerosis, primary biliary cirrhosis, Wegener's granulomatosis, and the inflammatory seguelia of infection.

Autoimmune disease often has an inflammatory component. Such conditions include acute disseminated alopecia universalise, ANCA positive diseases, Behcet's disease, Chagas' disease, chronic fatigue syndrome, dysautonomia, encephalomyelitis, ankylosing spondylitis, apiastic anemia, hidradenitis suppurative, autoimmune hepatitis, autoimmune oophoritis, celiac disease, inflammatory bowel disease, Crohn's disease, diabetes mellitus type 1, Fanconi syndrome, giant cell arteritis, glomerulonephritis.

Goodpasture's syndrome, Grave's disease, Guillain-Barre syndrome, Hashimoto's disease, Henoch-Schönlein purpura, Kawasaki's disease, systemic lupus erythematosus, microscopic colitis, microscopic polyarteritis, mixed connective tissue disease, multiple sclerosis, myasthenia gravis, opsocionus myoclonus syndrome, optic neuritis, Ord's thyroiditis, pemphigus, polyarteritis nodosa, polymyalgia, rheumatoid arthritis, Reiter's syndrome, Sjogren's syndrome, temporal arteritis, Wegener's granulomatosis, warm autoimmune haemolytic anemia, interstitial cystitis, lyme disease, morphea, psoriasis, sarcoidosis, scleroderma, ulcerative colitis, and vitiligo.

Other inflammatory conditions which may be treated with the compounds of the invention include, for example, appendicitis, dermatitis, dermatomyositis, endocarditis, fibrositis, gingivitis, glossitis, hepatitis, hidradenitis suppurative, iritis, laryngitis, mastitis, myocarditis, nephritis, otitis, pancreatitis, parotitis, pericarditis, peritonoitis, pharyngitis, pleuritis, pneumonitis, prostatistis, pyelonephritis, and stomatisi, transplant rejection (involving organs such as kidney, liver, heart, lung, pancreas (e.g., islet cells), bone marrow, cornea, small bowel, skin allografts, skin homografts, and heart valve xengrafts, sewrurn sickness, and graft vs host disease), acute pancreatitis, chronic pancreatitis, acute respiratory distress syndrome, Sexaiy's syndrome, congenital adrenal hyperplasis, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, astopic dermatitis, drug hypersensistivity reactions, allergic conjunctivitis, keratitis, herpes zoster ophthalmicus, iritis and oiridocyclitis, chonoretinitis, optic neuritis, symptomatic sarcoidosis, fulminating or disseminated pulmonary tuberculosis chemotherapy, idiopathic thrombocytopenic purpura in adults, secondary thrombocytopenia in adults, acquired (autoimmune) haemolytic anemia, leukaemia and lymphomas in adults, acute leukaemia of childhood, regional enteritis, autoimmune vasculitis, multiple sclerosis, chronic obstructive pulmonary disease, solid organ transplant rejection, sepsis, primary biliary cirrhosis and primary sclerosing cholangitis.

Preferred treatments using compounds of the invention include treatment of transplant rejection, rheumatoid arthritis, psoriatic arthritis. Type 1 diabetes, asthma, inflammatory bowel disease, systemic lupus erythematosis, and inflammation accompanying infectious conditions (e.g., sepsis), psoriasis, Crohns disease, ulcerative colitis, chronic obstructive pulmonary disease, multiple sclerosis, atopic dermatitis, and graft versus host disease.

Another preferred use of the compounds of the invention is in the treatment of cancers.

It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will be determined by clinical trial. However, it is expected that a typical dose will be in the range from about 0.001 to 50 mg per kg of body weight.

The compounds with which the invention is concerned may be prepared for administration by any route consistent with their pharmacokinetic properties. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol: preservatives, for example methyl or propyl p-hydroxybenzoate or sorbic acid, and if desired conventional flavouring or colouring agents.

For topical application to the skin, the drug may be made up into a cream, lotion or ointment. Cream or ointment formulations which may be used for the drug are conventional formulations well known in the art, for example as described in standard textbooks of pharmaceutics such as the British Pharmacopoeia.

For topical application by inhalation, the drug may be formulated for aerosol delivery for example, by pressure-driven jet atomizers or ultrasonic atomizers, or preferably by propellant-driven metered aerosols or propellant-free administration of micronized powders, for example, inhalation capsules or other “dry powder” delivery systems. Excipients, such as, for example, propellants (e.g. Frigen in the case of metered aerosols), surface-active substances, emulsifiers, stabilizers, preservatives, flavorings, and fillers (e.g. lactose in the case of powder inhalers) may be present in such inhaled formulations. For the purposes of inhalation, a large number of apparata are available with which aerosols of optimum particle size can be generated and administered, using an inhalation technique which is appropriate for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhalers for example as described in European Patent Application EP 0 505 321).

For topical application to the eye, the drug may be made up into a solution or suspension in a suitable sterile aqueous or non aqueous vehicle. Additives, for instance buffers such as sodium metabisulphite or disodium edeate; preservatives including bactericidal and fungicidal agents such as phenyl mercuric acetate or nitrate, benzalkonium chloride or chlorhexidine, and thickening agents such as hypromellose may also be included.

The active ingredient may also be administered parenterally in a sterile medium. Depending on the vehicle and concentration used, the drug can either be suspended or dissolved in the vehicle. Advantageously, adjuvants such as a local anaesthetic, preservative and buffering agent can be dissolved in the vehicle.

Compounds of the invention may be prepared, for example, by the methods described below and in the Examples herein.

Synthesis

There are multiple synthetic strategies for the synthesis of the compounds (I) with which the present invention is concerned, but all rely on known chemistry, known to the synthetic organic chemist. Thus, compounds according to formula (I) can be synthesised according to procedures described in the standard literature and are well-known to the one skilled in the art. Typical literature sources are “Advanced organic chemistry”, 4^{th} Edition (Wiley), J March; “Comprehensive Organic Transformation”, 2^{nd} Edition (Wiley), R. C. Larock; “Handbook of Heterocyclic Chemistry”, 2^{nd} Edition (Pergamon), A. R. Katritzky; review articles such as found in “Synthesis”, “Acc. Chem. Res:”, “Chem. Rev”, or primary literature sources identified by standard literature searches online or from secondary sources such as “Chemical Abstracts” or “Beilstein”. The synthetic routes used in the preparation of the compounds of the Examples below may be adapted for the preparation of analogous compounds.

### Abbreviations

MeOH=methanol
EtOH=ethanol
EtOAc=ethyl acetate
Boc=tert-butoxycarbonyl
DCM=dichloromethane
DMF=dimethylformamide
DCE=1,2-dichloroethane
TMSOK=potassium trimethylsilanoside
DMSO=dimethyl sulfoxide
TFA=trifluoroacetic acid
THE=tetrahydrofuran
Na₂CO₃=sodium carbonate
K₂CO₃=potassium carbonate
HCl hydrochloric acid
aq=aqueous solution
sat=saturated
DIPEA=diisopropylethylamine
NaH=sodium hydride
NaOH=sodium hydroxide
STAB=sodium triacetoxyborohydride
NaCNBH₃=sodium cyanoborohydride
NaHCO₃=sodium hydrogen carbonate
Pd/C=palladium on carbon
TBME=tert-butyl methyl ether
TPAP=tetrapropyl ammonium perruthenate
(COCl)₂=oxalyl chloride
N₂=nitrogen
PyBop benzotriazole-1-yl-oxy-tris-pyrrolidino-phosphonium hexafluorophosphate
Na₂SO₄=sodium sulphate
Et₃N=triethylamine
NH₃=ammonia
TMSCl trimethylchlorosilane
NH₄Cl=ammonium chloride.
LiAlH₄=lithium aluminium hydride
PyBrOP Bromo-tris-pyrrolidino phosphoniumhexafiuorophosphate
MgSO₄=magnesium sulfate
ⁿBuLi=n-butyllithium
CO₂=carbon dioxide
EDCl=N-(3-Dimethylaminopropyl)-N′-ethylcarbodiimide hydrochloride
Et₂O=diethyl ether
LiOH=lithium hydroxide
HOBt=1-hydroxybenzotriazole
TLC=thin layer chromatography
LCMS=liquid chromatography/mass spectrometry
mL=millilitre(s)
g=gram(s)
mg=milligram(s)
mol=mob(s)
mmol=millimole(s)
HPLC=high performance liquid chromatography
NMR=nuclear magnetic resonance
RT=room temperature
h=hour(s)

Commercially available reagents and solvents (HPLC grade) were used without further purification. Solvents were removed using a Buchi rotary evaporator. Microwave irradiation was carried out using a Biotage Initiator™ Eight microwave synthetiser. Purification of compounds by flash column chromatography was performed using silica gel, particle size 40-63μ μm (230-400 mesh) obtained from Fluorochem. Reverse phase column chromatography was performed using a pre-column on Merck IiChroprep RP-18 (40-60 μm) before purification on a CombiFlash Companion (Teledyne Isco, Nebraska, USA) using RediSep Rf C18 columns (Presearch, Basingstoke, UK). Purification of compounds by preparative HPLC was performed on Gilson systems using reverse phase Axia™ prep Luna C18 columns (10 μmu, 100×21.2 mm), gradient 0-100% B (A=water/0.05% TFA, B=acetonitrile/0.05% TFA) over 10 min, flow=25 ml/min, UV detection at 254 nm.

¹H NMR spectra were recorded on a Bruker 300 MHz AV spectrometer in deuterated solvents. Chemical shifts (δ) (δ) are in parts per million. Thin-layer chromatography (TLC) analysis was performed with Kieselgel 60 F₂₅₄ (Merck) plates and visualized using UV light.

Analytical HPLC/MS was performed on an Agilent HP1100 LC system using reverse phase Luna C18 columns (3μ μm, 50×4.6 mm), gradient 5-95% B (A=water/0.1% Formic acid, B acetonitrile/0.1% Formic acid) over 2.25 min, flow=2.25 ml/min. UV spectra were recorded at 220 and 254 nm using a G1315B DAD detector. Mass spectra were obtained over the range m/z 150 to 800 on a LC/MSD SL G1956B detector. Data were integrated and reported using ChemStation and ChemStation Data Browser softwares.

Thus, compounds of general formula (8) and (9) may be, but not exclusively, prepared by the methods outlined in Scheme 1.

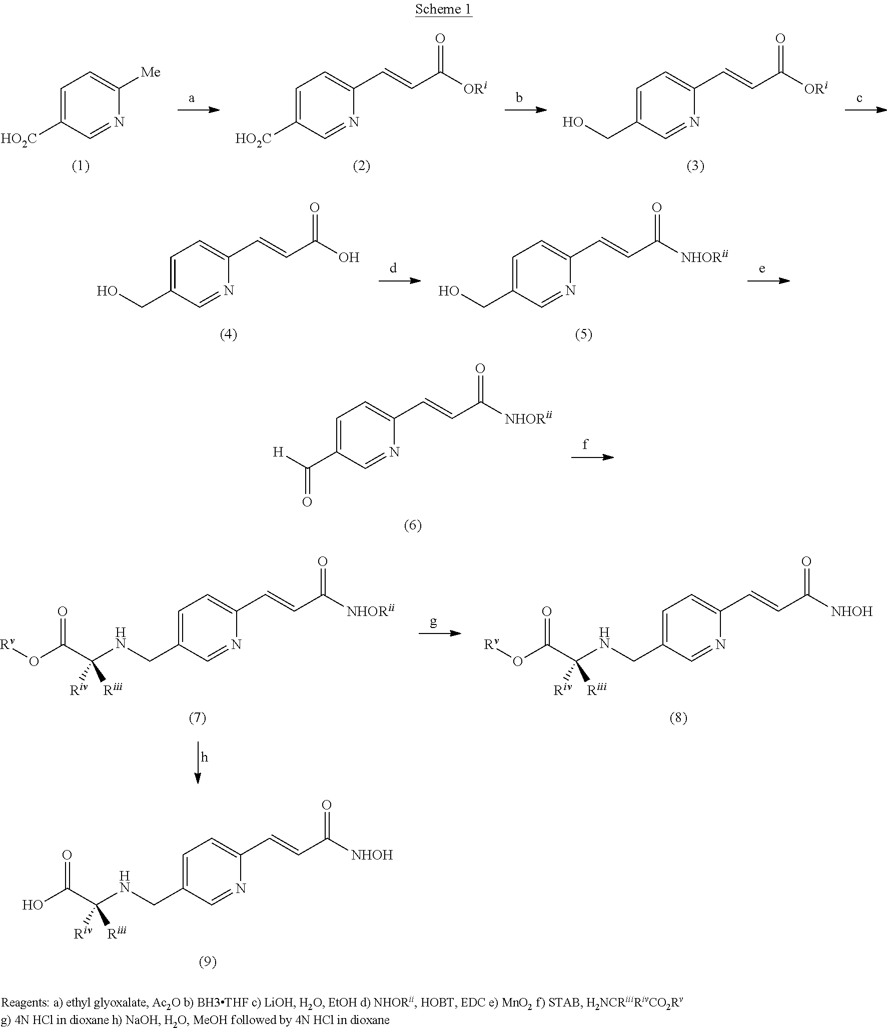

Thus heteroaromatic carboxylic acids such as 8-methylnicotinic acid (1) may be used in a condensation reaction with aldehydic reagents such as ethyl glyoxalate in the presence of acetic anhydride in hydrocarbon solvents such as toluene under reflux conditions to give α,β-unsaturated esters of general formula (2). The carboxylic substituent of (2) may be transformed to a hydroxymethylene group by the use of reducing agents such as borane THF complex to give alcohols of general formula (3). α,β-Unsaturated acids of general formula (4) may be obtained from (3) under basic hydrolysis conditions employing an alkali such as sodium or lithium hydroxide in the presence of a water miscible co-solvent such as methyl or ethyl alcohol. O-Protected hydroxamic acids of general formula (5) may be prepared by the coupling of protected hydroxylamines such as O-(1-isobutoxyethyl)hydroxylamine (WO 01/60785) using reagents such as N-hydroxybenzotriazole and 1-ethyl-3-(3-dimethylaminoprepyl) carbodiimide hydrochloride or N,N′-diisopropylcarbodiimide. Oxidation of compounds of general formula (5) to the corresponding aldehydes may be performed by the use of reagents such as manganese dioxide. Reductive amination of aldehydes such as (6) with α,β-disubstituted amino acid esters using reagents such as sodium borohydride, sodium cyanoborohydride or sodium triacetoxyborohydride leads to amino acid ester derivatives of general formula (7). Hydroxamic acids of general formula (8) may be prepared by the treatment of (7) under acidic conditions such as hydrochloric acid in solvents such as 1,4-dioxane. Amino acid derivatives of general formula (9) may be prepared by the hydrolysis of (7) under aqueous alkaline conditions using for example aqueous sodium hydroxide in the presence of a water miscible co-solvent such as methyl alcohol or tetrahydrofuran.

Alternatively compounds of general formula (8) may be prepared by methods described in Scheme 2.

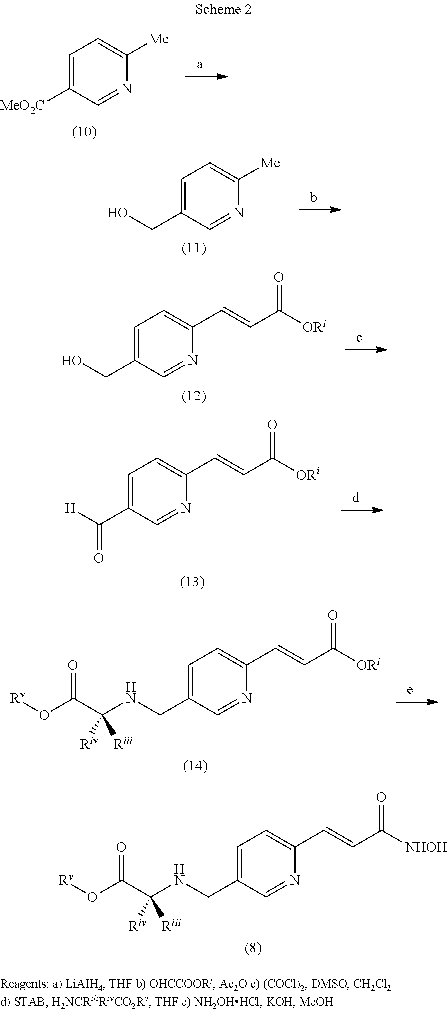

Thus compounds such as methyl-6-methylnicotinate (10) may be reduced with hydride donors such as lithium aluminium hydride to give alcohols such as (11) which possess an activated alkyl group which can be utilized in condensation reactions with aldehydes such as ethyl glyoxalate to give α,β-unsaturated esters such as (12). Compounds such as (12) may be further oxidized under conditions such as those described by Swern [J. Org. Chem. 1976, 41, 3329] employing, for example, oxalyl chloride and DMSO to give aldehydes of general formula (13). In turn aldehydes such as (13) may be converted to amino acid esters of formula (14) by reductive amination procedures such as those described by Borch [J Am. Chem. Soc. 1969, 91, 3006] employing cyanoborohydride or triacetoxyborohydride anions. Hydroxamic acids of formula (8) may be prepared by the reaction of compounds of formula (14) with hydroxylamine hydrochloride in the presence of an alkali such as sodium or potassium hydroxide.

Compounds of general formula (21) and (23) may be, but not exclusively, prepared by methods outlined in Scheme 3.

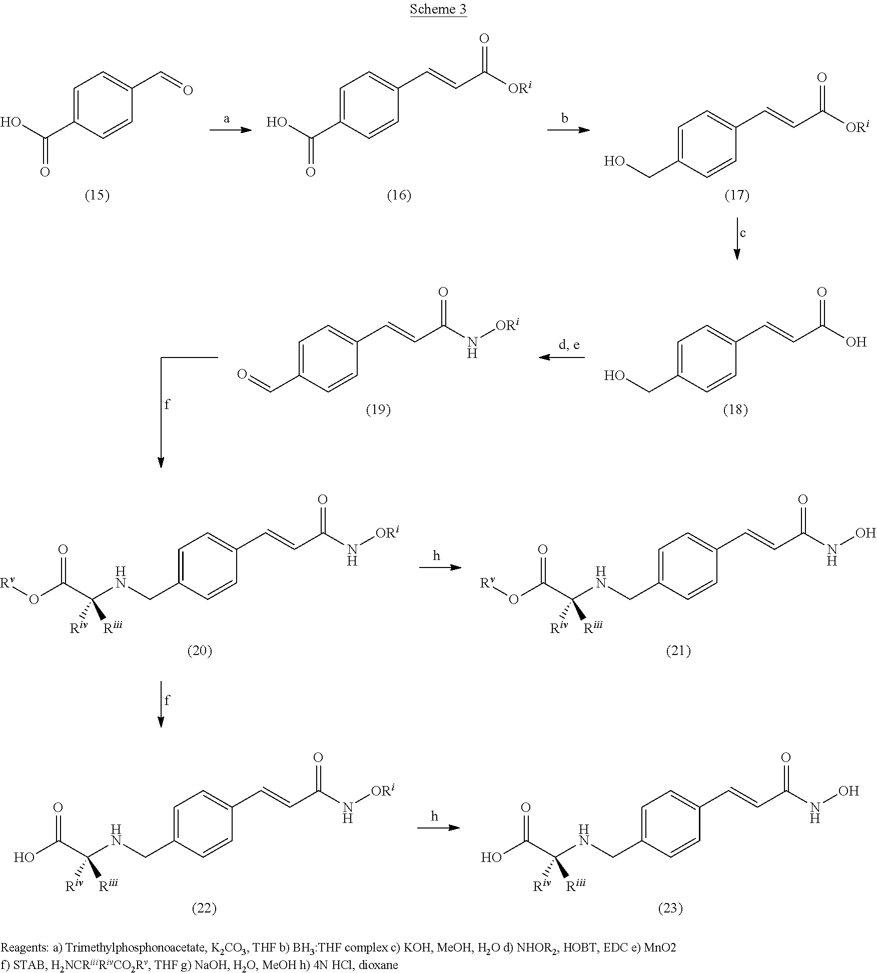

Thus α,β-unsaturated esters such as compounds of general formula (16) may be prepared by a Homer-Emmons reaction between a phosphonate carbanion and an aldehyde such as (15) in the presence of an inorganic base such as potassium carbonate under aqueous conditions. Alternatively other bases such as sodium hydride in DMSO or organic bases such as DBU in acetonitrile could be employed for this transformation. Alcohols of general formula (17) can be obtained by reduction of acids such as (16) with hydride-donor reagents such as borane in inert solvents such as THF. Hydrolysis of esters of general formula (17) to acids of general formula (18) may be performed by a mineral base such as sodium or potassium hydroxide under aqueous conditions in the presence of a co-solvent such as methanol. Aldehydes of general formula (19) may be obtained from (18) by a coupling reaction with an O-protected hydroxylamine in the presence of reagents such as N-hydroxybenzotriazole and 1-ethyl-3-(3-dimethylaminopropyl) carbodiimide hydrochloride or N,N′-diisopropyl carbodiimide followed by oxidation of the alcohol substituent of the resulting hydroxamic intermediate with a reagent such as manganese dioxide, Aldehydes of formula (19) may then be reacted with amino acid esters under conditions of reductive amination with reagents such as sodium triacetoxyborohydricle or sodium cyanoborohydride to give compounds of general formula (20), Hydroxamic acids of general formula (21) may be prepared by deprotection of compounds of type (20), for example where R² is (1-isobutoxyethyl), with acidic reagents such as 4M HCl in dioxane. Amino acids such as (22) may be prepared by treating compounds of general formula (20) with a mineral base such as lithium hydroxide, Hydroxamic acids of general formula (23) may be prepared by treating compounds of formula (22) under acid conditions, for example with hydrochloric acid.

Alternatively compounds such as (23) may be obtained by a process such as described in Scheme 4. Thus reagents such as 4-diethoxybenzaldehyde (24) may be reacted with trialkylphosphonoacetates in the presence of salts such as lithium bromide and organic bases such as triethylamine to give aldehydes such as (25) after acid work up. In turn aldehydes such as (25) may be converted to amino acid esters of formula (26) by reductive amination procedures such as those described by Borch [J Am. Chem. Soc. 1969, 91, 3006] employing cyanoborohydride or triacetoxyborohydride anions, Hydroxamates of general formula (23) may then be prepared by the treatment of compounds such as (26) with hydroxylamine hydrochloride in the presence of base such as potassium or lithium hydroxide in a solvent such as methanol or ethanol.

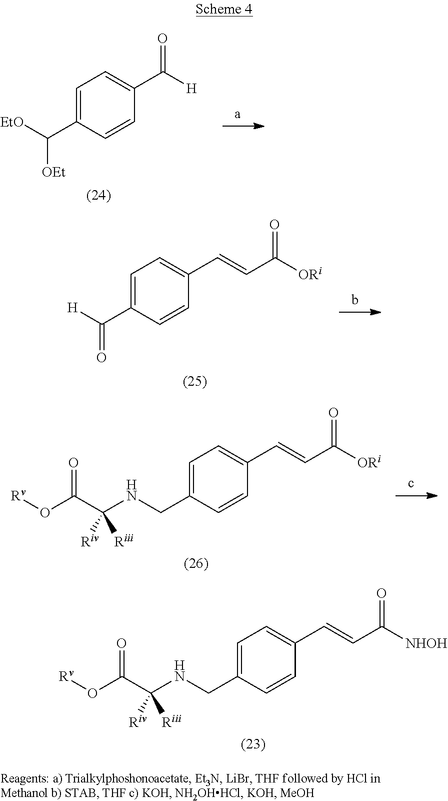

Amino acid derivatives of general formula (28) may also be prepared by methods described in Scheme 5.

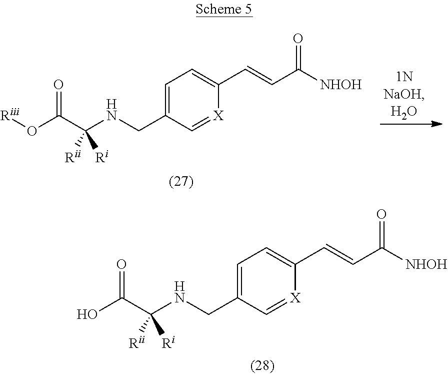

Thus esters of general formula (27) [X=CH or N] may be hydrolysed to acids of type (28) [X=CH,N] with alkaline bases such as potassium or sodium hydroxide. In another procedure acids of general formula (28) [X=CH,N] may be prepared by methods described in Scheme 6. Thus aldehydes of general formula (29) [X=CH,N] and amino acids are reacted under conditions of reductive amination to yield intermediates of general formula (30) [X=CH,N]. The protected hydroxamates of formula (30) [X=CH,N] are treated under acid conditions, such as with hydrochloric acid to give acids of general formula (28) [X=CH,N].

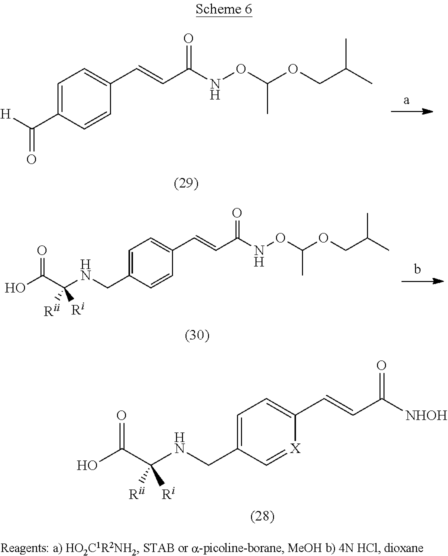

Amino acid esters of general formula (32) may be prepared by a number of methods including those described in Scheme 7. Thus amino acids of formula (31) may be heated with the appropriate alcohol (R³OH) in the presence of H₂SO₄ or reacted with the appropriate alcohol (R³OH) under Dean-Stark conditions in the presence of an acid such as para-toluenesulphonic acid to give esters of formula (32).

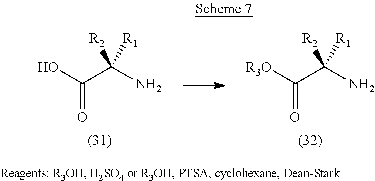

### INTERMEDIATES

### Intermediate 1 Ethyl (2E)-3-(5-formylpyridin-2-yl)acrylate

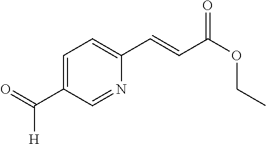

Stage 1

Lithium aluminium hydride (23 g, 1.2 eq) in THF (500 mL) was cooled to −78° C. Methyl-6-methylnicotinate was dissolved in THF (200 mL) and charged to the reaction at below −70° C. The reaction was allowed to warm to 0° C. over 1 h and aged at 0+ C. for 1 h. On completion the reaction was quenched with sat. NaHCO₃ (250 mL) below 10° C. The reaction mixture was filtered to remove inorganics, the filter cake was washed with THF and the filtrate concentrated in vacuo to remove most of the THF. The residue was separated between ethyl acetate and water, the aqueous layer being extracted three times with EtOAc. Combined organics washed with K₂CO₃(aq), dried (MgSO₄) then concentrated to dryness to afford the product (40.5 g). ¹H NMR (CDCl3): (8.35, 1H, s), 7.61 (1H, dd), 7.13 (1H, d), 4.65 (2H, d), 2.51 (3H, d). A second extraction of the aqueous layer provided additional product (5.5 g) which was combined with the product from the first extraction.

Stage 2

The product from Stage 1 (46 g, 1 eq) was dissolved in acetic anhydride (670 mL) and stirred at 80° C. for approximately 1 h. Ethyl glyoxal solution (50% in toluene) (147 mL, 2 eq) was charged to the reaction vessel which was then heated for overnight at 100° C. An additional amount of ethyl glyoxal solution (50% in toluene) (37 mL, 0.5 eq) was added at approximately 16 h after reflux was initiated and heating was continued for 2 h. The reaction was quenched with water (100 mL), stirred at ˜50° C. for 40 min then concentrated in vacuo. The residue was basified to pH 9-10 with 1N NaOH and then solid NaOH. After extraction twice with EtOAc, the combined organics were washed with 0.25N NaOH then concentrated to dryness. The crude residue was stirred in a mixture of ethanol (500 mL) and conc. HCl (50 mL) for overnight at 40° C. On completion of deacetylation the reaction mixture was concentrated to dryness, separated between EtOAc and K₂CO₃(aq) and the aqueous phase extracted with EtOAc. The combined organics were washed with K₂CO₃(aq), dried (MgSO₄), then concentrated to dryness in vacuo. Purification by dry-flash chromatography (3:2-1:4 heptanes:EtOAc eluant) afforded the desired product (21.9 g). ¹H NMR (CDCl3): 8.64 (1H, s), 7.76-7.79 (1H, m),

Stage 3

DCM (20 mL) and DMSO (3.4 mL, 5 eq) were charged to a flask and cooled to below −70° C. Oxalyl chloride (1.47 mL, 2.2 eq) was charged drop-wise at below −65° C. then the reaction aged for ˜0.5 h. The Stage 2 product (2 g, 1 eq) was dissolved in DCM (20 mL) and charged to the reaction which was then aged at below −70° C. for ˜1 h. Triethylamine (6.7 mL, 5 eq) was charged and the reaction allowed to warm to ambient temperature. Water (40 mL) was charged to the reaction vessel, the layers separated and the aqueous phase extracted with DCM. The combined organics were washed with water, dried (MgSO₄) then concentrated to dryness to afford Intermediate 1 (1.7 g). ¹H NMR (300 MHz, CDCl₃) δ (ppm): 10.14 (1H, s), 9.10 (1H, d), 8.20 (1H, dd), 7.72 (1H, d), 7.60 (1H, d)

### Intermediate 2 (2E)-3-(5-Formylpyridin-2-yl)-N-(1-isobutoxyethoxy)acrylamide

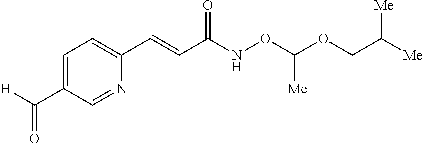 [SMILES: CC(C)COC(C)ONC(=O)/C=C/c1ccc(C=O)cn1]

Intermediate 2 was prepared by methods described in WO2008/040934.

### Intermediate 3 (2E)-3-(4-Formylphenyl)-N-(1-isobutoxyethoxy)acrylamide

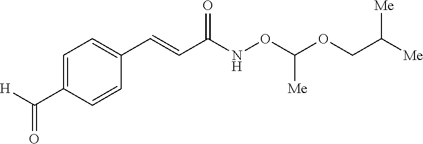 [SMILES: CC(C)COC(C)ONC(=O)/C=C/c1ccc(C=O)cc1]

Intermediate 3 was prepared by methods described in WO2008/040934.

### Intermediate 4 3-(4-Formylphenyl)-N-(1-isobutoxyethoxy)propanamide

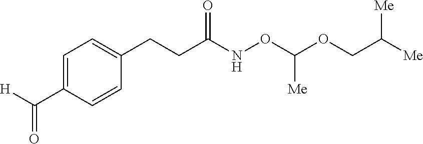 [SMILES: CC(C)COC(C)ONC(=O)CCc1ccc(C=O)cc1]

Intermediate 4 was prepared by methods described in WO2008/040934.

### Intermediate 5 Cyclopentyl 1-[({6-[(1E)-3-ethoxy-3-oxoprop-1-en-1-yl]pyridin-yl}methyl)amino]cyclobutanecarboxylate

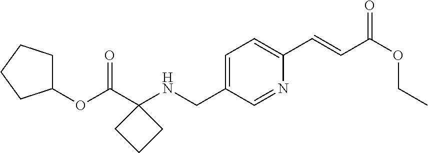 [SMILES: CCOC(=O)/C=C/c1ccc(CNC2(C(=O)OC3CCCC3)CCC2)cn1]

STAB (1.2 g, 5.6 mmol) was charged to a slurry of Intermediate 1 (0.77 g, 3.75 mmol) and Intermediate 20 (1.25 g, 3.74 mmol) in THF (15 mL). The reaction was stirred for 16 h at ambient temperature then quenched with sat. NaHCO₃(20 mL). The layers were separated and the aqueous phase extracted with EtOAc (20 mL). The combined organic phases were dried (MgSO₄), concentrated to dryness then the residue purified by silica chromatography to afford the title compound as a yellow oil (0.75 g), m/z 373 [M+H]⁺

The following Intermediates were prepared in a manner similar to Intermediate 5

### Intermediate 6 Cyclopentyl 1-{[(6-{(1E)-3-[(1-isobutoxyethoxy)amino]3-oxoprop-1-en-1-yl}pyridin-3-yl)methyl]amino}cyclopentanecarboxylate

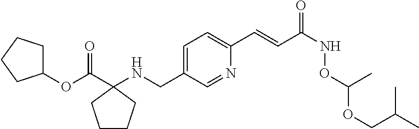 [SMILES: CC(C)COC(C)ONC(=O)/C=C/c1ccc(CNC2(C(=O)OC3CCCC3)CCCC2)cn1]

From Intermediate 2 (100 mg, 0.34 mmol) and Intermediate 19 (tosylate salt) (125.5 mg, 0.34 mmol) to give Intermediate 6 (84.7 mg), m/z 474 [M+H]⁺

### Intermediate 7 Cyclopentyl 1-[({6-[(1E)-3-ethoxy-3-oxoprop-1-en-1-yl]pyridin-3-yl}methyl)amino]cyclohexanecarboxylate

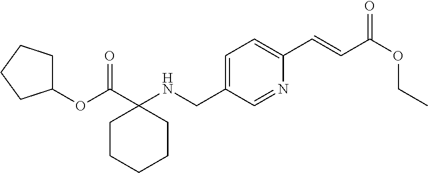 [SMILES: CCOC(=O)/C=C/c1ccc(CNC2(C(=O)OC3CCCC3)CCCCC2)cn1]

From Intermediate 1 (0.1 g) and Intermediate 18 (0.1 g) to give Intermediate 7 (60 mg), m/z 400 [M+H]⁺

### Intermediate 8 Cyclopentyl 1-({4-[(1E)-3-methoxy-3-oxoprop-1-en-1-yl]benzyl}amino)cyclopentanecarboxylate Hydrochloride

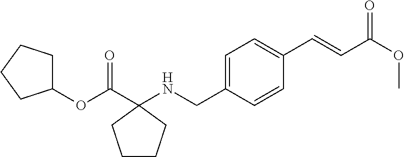 [SMILES: COC(=O)/C=C/c1ccc(CNC2(C(=O)OC3CCCC3)CCCC2)cc1]

From Intermediate 29 (4.4 g, 23 mmol) and Intermediate 19 (tosylate) (8 g, 23 mmol) to give Intermediate 8 (7.33 g) as the hydrochloride salt by treating a solution of the free base (8.7 g) in ethyl acetate (90 mL) with 2N HCl in diethyl ether (11.7 mL). ¹H NMR (300 MHz, d₆-DMSO) δ (ppm): 9.99 (2H, bs), 7.81 (2H, d), 7.69 (1H, d), 7.61 (2H, d), 6.72 (1H, d), 5.23 (1H, m), 4.13 (2H, m), 3.72 (3H, s), 2.30-1.50 (16H, m).

### Intermediate 9 Cyclopentyl 1-({4-[(1E)-3-methoxy-3-oxoprop-1-en-1-yl]benzyl}amino)cyclobutanecarboxylate Hydrochloride

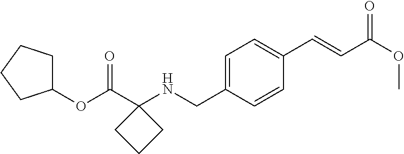 [SMILES: COC(=O)/C=C/c1ccc(CNC2(C(=O)OC3CCCC3)CCC2)cc1]

From Intermediate 29 (0.57 g, 2.9 mmol) and Intermediate 20 (1 g, 2.6 mmol) to give Intermediate 9 (0.63 g) as the hydrochloride salt by treating a solution of the free base (1.15 g) in ethyl acetate (15 mL) with 2N HCl in diethyl ether (1.55 mL). The product was collected by filtration, after ageing in an ice bath. The resulting solid was washed with ethyl acetate and dried. m/z 358 [M+H]⁺

### Intermediate 10 Cyclopentyl N-{4-[(1E)-3-methoxy-3-oxoprop-1-en-1-yl]benzyl}-2-methyl-D-alaninate

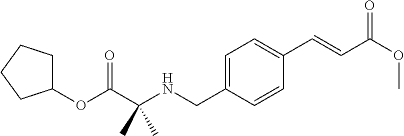

From intermediate 29 (360 mg, 1.05 mmol) and Intermediate 28 (200 mg, 1.05 mmol) to give Intermediate 10 (103.9 mg). m/z 346 [M+H]⁺

### Intermediate 11 Cyclopentyl 1-[(4-{(1E)-3-[(1-isobutoxyethoxy)amino]-3-oxoprop-1-en-1-yl}benzyl)amino]cyclopropanecarboxylate

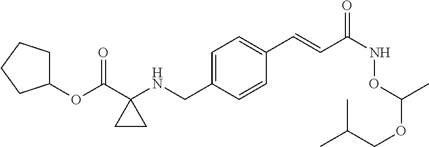 [SMILES: CC(C)COC(C)ONC(=O)/C=C/c1ccc(CNC2(C(=O)OC3CCCC3)CC2)cc1]

From intermediate 3 (100 mg, 0.34 mmol) and Intermediate 23 (57.5 mg, 0.34 mmol) to give Intermediate 11 (59 mg). m/z 445 [M+H]⁺

### Intermediate 12 Cyclopentyl 1-[4-{(1E)-3-[1-isobutoxyethoxy)amino]-3-oxoprop-1-en-1-yl}benzyl)amino]cyclohexanecarboxylate

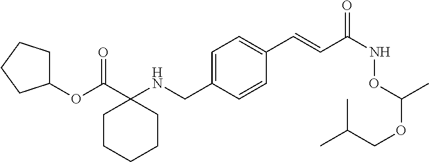 [SMILES: CC(C)COC(C)ONC(=O)/C=C/c1ccc(CNC2(C(=O)OC3CCCC3)CCCCC2)cc1]

From Intermediate 3 (100 mg, 0.34 mmol) and Intermediate 18 (71.7 mg, 0.34 mmol) to give Intermediate 12 (64.9 mg). m/z 487 [M+H]⁺

### Intermediate 13 Cyclopentyl N-(4-{(1E)-3-[(1-isobutoxyethoxy)amine]-3-oxoprop-1-en-1-yl}benzyl)-α-methyl-L-phenylalaninate

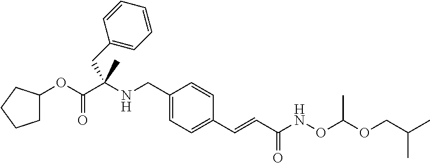 [SMILES: CC(C)COC(C)ONC(=O)/C=C/c1ccc(CN[C@@](C)(Cc2ccccc2)C(=O)OC2CCCC2)cc1]

From Intermediate 3 (100 mg, 0.34 mmol) and Intermediate 27 (84 mg, 0.34 mmol) to give Intermediate 13 (83 mg). m/z 523 [M÷H]⁺

### Intermediate 14 Cyclopentyl N-(4-{(1E)-3-[(1-isobutoxyethoxy)amino]-3-oxoprop-1-en-1-yl}benzyl)-2-methyl-D-leucinate

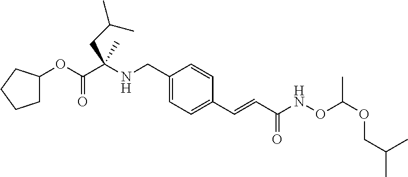 [SMILES: CC(C)COC(C)ONC(=O)/C=C/c1ccc(CN[C@](C)(CC(C)C)C(=O)OC2CCCC2)cc1]

From Intermediate 3 (100 mg, 0.34 mmol) and intermediate 25 (72.4 mg, 0.34 mmol) to give intermediate 14 (78.2 mg). m/z 489 [M+H]⁺

### Intermediate 15 Cyclopentyl N-(4-{(1E)-3-[(1-isobutoxyethoxy)amino]-3-oxoprop-1-en-1-yl}benzyl)-2-methyl-L-leucinate

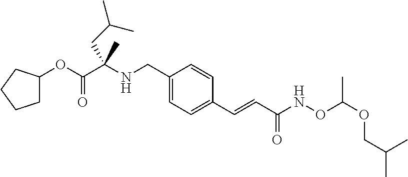 [SMILES: CC(C)COC(C)ONC(=O)/C=C/c1ccc(CN[C@@](C)(CC(C)C)C(=O)OC2CCCC2)cc1]

From Intermediate 3 (100 mg, 0.34 mmol) and intermediate 26 (72 mg, 0.34 mmol) to give Intermediate 15 (84.8 mg). m/z 489 [M+H]⁺

### Intermediate 16 Cyclopentyl N-(4-{(1E)-3-[(1-isobutoxyethoxy)amino]-3-oxoprop-1-en-1-yl}benzyl)-L-isovalinate

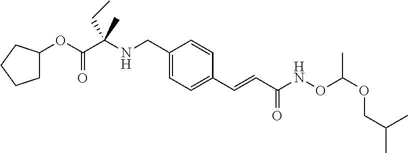 [SMILES: CC[C@](C)(NCc1ccc(/C=C/C(=O)NOC(C)OCC(C)C)cc1)C(=O)OC1CCCC1]

From Intermediate 3 (100 mg, 0.34 mmol) and intermediate 24 (63 mg, 0.34 mmol) to give Intermediate 16 (47 mg). m/z 461 [M+H]⁺

### Intermediate 17 Cyclopentyl N-(4-{(1E)-3-[(1-isobutyoxyethoxy)amino]-3-oxoprop-1-en-1-yl}benzyl)-3-methyl-L-isovalinate

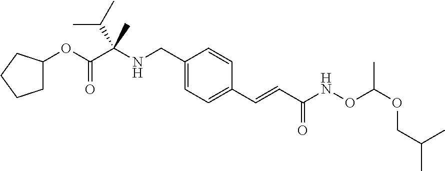 [SMILES: CC(C)COC(C)ONC(=O)/C=C/c1ccc(CN[C@](C)(C(=O)OC2CCCC2)C(C)C)cc1]

From Intermediate 3 (100 mg, 0.34 mmol) and Intermediate 22 (68 mg. 0.34 mmol) to give Intermediate 17 (75.7 mg). m/z 375 [M+H]⁺

### Intermediate 18 Cyclopentyl 1-aminocyclohexanecarboxylate tosylate

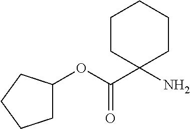 [SMILES: NC1(C(=O)OC2CCCC2)CCCCC1]

1-aminocyclohexanecarboxylic acid (4.2 g, 29 mmol) in cyclohexane (250 mL) was added cyclopentanol (50 mL) and para-toluenesulphonic acid (5.89 g) and the resulting suspension heated at reflux in a Dean-Stark apparatus for 72 h. On cooling to room temperature the resulting white solid was collected by filtration and washed with cyclohexane (2×100 mL) and dried under reduced pressure to give the Intermediate 18 (tosylate salt) (4.1 g) as a colourless solid. m/z 212.3 [M+H]⁺.

### Intermediate 19 Cyclopentyl 1-aminocyclopentanecarboxylate

### Method A

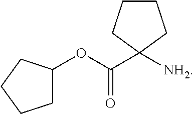 [SMILES: NC1(C(=O)OC2CCCC2)CCCC1]

To a solution of 1-aminocyclopentanecarboxylic acid (2.58 g, 19.97 mmol) in cyclopentanol (20 ml), was added concentrated sulfuric acid (2.15 g, 21.97 mmol) and the mixture stirred over night at 70° C. The reaction was allowed to cool to RT and the cyclopentanol removed under reduced pressure. The residue was dissolved in EtOAc (30 ml) and washed with sat. NaHCO₃(30 ml) and water (3×20 ml) then dried (MgSO₄), filtered and concentrated in vacuo to leave a dark yellow oil. Purification by column chromatography (15% 1.2M NH₃/MeOH in EtOAc) afforded Intermediate 19 (1.97 g).

¹H NMR (300 MHz, CDCl₃) δ (ppm): 5.21-5.17 (1H, m), 2.15-1.90 (2H, m), 1.85-1.57 (14H, m).

### Method B

Following a method similar to that of Intermediate 18 cyclopentyl 1-aminocyclopentane carboxylate tosylate (28.9 g, 147 mmol) was prepared from 1-aminocyclopentane carboxylic add (10.7 g) and cyclopentanol (37.5 mL) in the presence of para-toluenesulphonic acid (17.3 g).

### Intermediate 20 Cyclopentyl 1-aminocyclobutanecarboxylate tosylate

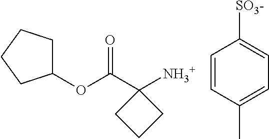

1-Amino-1-cyclobutanecarboxylic acid (1 g, 8.7 mmol), cyclopentanol (2.4 mL) and para-toluenesulphonic add (1.8 g, 9.6 mmol) were stirred in cyclohexane (5 mL) and heated to reflux for 23 h before additional cyclohexane (3 mL) was added as the reaction became dry. After an additional 80 minutes the reaction was cooled briefly to 70° C. and cyclohexane (5 mL) and cyclopentanol (2.4 mL) were added and heating at reflux was continued until the reaction was complete. On cooling the reaction to ambient temperature methyl t-butyl ether (50 mL) was added and the reaction stirred for 10 min. The resulting solid was collected by filtration and washed with methyl t-butyl ether (20 mL) to give Intermediate 20 (2.83 g) as the tosylate salt. m/z 184 [M+H]⁺ 1H NMR (300 MHz, d₆-DMSO) δ (ppm): 7.72 (2H, d), 7.25 (2H, d), 5.35 (1H, m), 2.58-2.67 (2H, m), 2.42-2.48 (2H, m), 2.41 (3H, s), 2.10-2.39 (2H, m), 1.93-2.08 (2H, m), 1.71-1.86 (6H, m)

The following Intermediates were prepared in a similar manner to Intermediate 19

### Intermediate 21 Cyclopentyl 2-methyl-D,L-leucinate

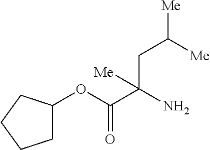 [SMILES: CC(C)CC(C)(N)C(=O)OC1CCCC1]

From (R,S)-α-methylleucine (500 mg, 3.44 mmol) to give Intermediate 21 (650 mg) m/z 214.3 [M+H]⁺.

### Intermediate 22 Cyclopentyl 3-methyl-L-isovalinate

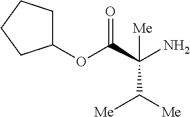 [SMILES: CC(C)[C@](C)(N)C(=O)OC1CCCC1]

From 3-methyl-L-isovaline (500 mg) to give Intermediate 22 (292 mg) as an orange oil. m/z 200.2 [M+H]⁺.

### Intermediate 23 Cyclopentyl 1-aminocyclopropanecarboxylate

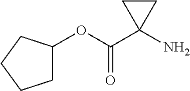 [SMILES: NC1(C(=O)OC2CCCC2)CC1]

From 1-aminocyclopropane-1-carboxylic acid (500 mg, 4.95 mmol) to give intermediate 23 (302.9 mg) m/z 170 [M+H]⁺

### Intermediate 24 Cyclopentyl L-isovalinate

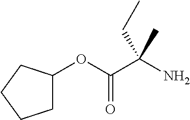

From (S)-α-ethylalanine (1 g, 8.54 mmol) to give Intermediate 24 (1.03 g) m/z 186 [M+H]⁺

### Intermediate 25 Cyclopentyl 2-methyl-D-leucinate

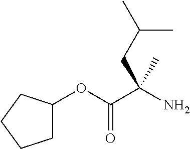

From (R)-α-methylleucine (1.0 g, 8.9 mmol) to Intermediate 25 (1.12 g) m/z 214 [M+H]⁺

### Intermediate 26 Cyclopentyl 2-methyl-L-leucinate

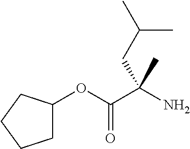

From (S)-α-methylleucine (1.0 g, 6.9 mmol) to give Intermediate 26 (0.61 g), m/z. 214 [M+H]⁺

### Intermediate 27 Cyclopentyl α-methyl-L-phenylalaninate

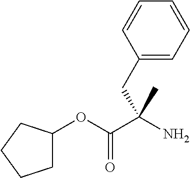 [SMILES: C[C@](N)(Cc1ccccc1)C(=O)OC1CCCC1]

From (S)-α-methylphenylalanine (1.0 g, 5.58 mmol) to give Intermediate 27 (0.62 g), m/z 248 [M+H]⁺

### Intermediate 28 Cyclopentyl 2-methylalaninate Hydrochloride

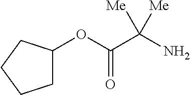 [SMILES: CC(C)(N)C(=O)OC1CCCC1]

### Stage 1

To a solution of N-(tert-butoxycarbonyl)-2-methylalanine (1.00 g, 4.92 mmol) DCM (10 ml) at 0° C. was added cyclopentanol (0.83 ml, 9.84 mmol), EDCl (1.06 g, 5.42 mmol) and finally DMAP (60 mg, 0.49 mmol). The reaction mixture was warmed to RT and stirred for 18 h The DCM was removed in vacuo to give a clear oil. The crude residue was dissolved in EtOAc (100 ml) and washed with water, 1M NaHCO₃ and brine. The organic phase was dried (MgSO₄) and concentrated in vacuo. The crude extract was purified by column chromatography (10% EtOAc in heptane) to yield the desired product as a clear oil (0.254 g, 20% yield).

¹H NMR (300 MHz, CDCl₃) δ (ppm): 5.25-5.17 (1H, m), 5.04 (1H, br s), 1.93-1.54 (8H, m),

### Stage 2

Cyclopentyl N-(tert-butoxycarbonyl)-2-methylalaninate (0.254 g, 0.93 mmol) was dissolved in THF (5 ml) and treated with 4M HCl in dioxane (2 ml) and the reaction mixture was stirred at RT for 24 hrs. The crude mixture was concentrated under reduced pressure and triturated with Et₂O to give a white precipitate. This was further washed with Et₂O to give Intermediate 28 as a white powder (0.16 g, 82% ¹H NMR (300 MHz, DMSO-d₆) δ (ppm): 8.58 (3H, hr s), 5.21-5.14 (1H, m), 1.93-1.78 (2H, m), 1.74-1.53 (6H, m), 1.45 (6H, s).

### Intermediate 29 Methyl (2E)-3-(4-formylphenyl)acrylate

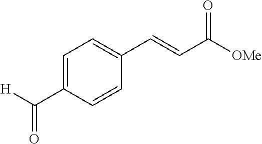 [SMILES: COC(=O)/C=C/c1ccc(C=O)cc1]

Lithium bromide (159 g, 2.5 eq) was dissolved in THF (2 L) and cooled to <5° C. Trimethyl phosphonoacetate (1.3 eq, 138 mL) then triethylamine (204 mL, 2 eq) were charged at <10° C. 4-Diethoxybenzaldehyde (152.8 g, leg) was charged over 25 mins and the reaction allowed to warm to 20±5° C., then the cooling was removed. After 1 h 40 min, the reaction was quenched with water, separated, and the aqueous layer extracted with EtOAc. The combined organic phases were washed three times with brine then concentrated to dryness in vacuo. Methanol (0.5 L) was charged to the residue and again concentrated to dryness. Methanol (0.75 L) and 1N HCl (0.75 L) were charged to the residue and stirred at ambient temperature for 45 min. Water (0.75 L) was charged and the product isolated by filtration (128.5 g). ¹H NMR (300 MHz, CDCl₃) δ (ppm): 10.06 (1H, s), 7.93 (2H, d), 7.71 (3H, m), 6.58 (1H, d), 3.85 (3H, s).

### Intermediate 30 1-[(4-{(1E)-3-[(1-Isobutoxyethoxy)amino]-3-oxoprop-1-en-1-yl}benzyl)amino]cyclopentanecarboxylic acid

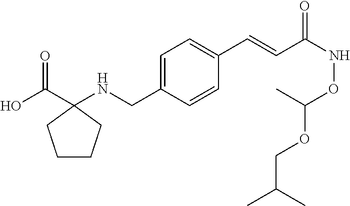 [SMILES: CC(C)COC(C)ONC(=O)/C=C/c1ccc(CNC2(C(=O)O)CCCC2)cc1]

Intermediate 3 (100 mg, 0.34 mmol) and 1-aminocyclopentanecarboxylic acid (48.3 mg, 0.37 mmol) were dissolved in methanol (10 mL) and stirred at room temperature for 1 h and α-picoline-borane (72.08 mg, 0.68 mmol) was then added. After 4 h, the reaction appeared to have gone to completion and the solvent was removed under reduced pressure. The residue was purified by chromatography (reverse phase silica, CH₃CN in water, gradient 0 to 100%) to give Intermediate 30 (13.6 mg). m/z 405 [M+H]⁺

### Intermediate 31 1-{[(6-{(1E)-3-[(1-isobutoxyethoxy)amino]-3-oxoprop-1-en-1-yl}pyridin-3-yl)methyl]amino}cyclopentanecarboxylic acid

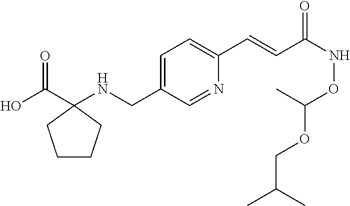 [SMILES: CC(C)COC(C)ONC(=O)/C=C/c1ccc(CNC2(C(=O)O)CCCC2)cn1]

In a manner similar to Intermediate 30 from Intermediate 2 (200 mg, 0.68 mmol), 1-aminocyclopentanecarboxylic acid (43.8 mg, 0.34 mmol) and α-picoline-borane (109 mg) to give Intermediate 31 (70 mg). m/z 406 [M+H]⁺

### Intermediate 32 t-butyl 2-methylalaninate

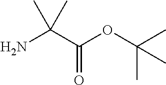

### Stage 1

To a solution of N-[(benzyloxy)carbonyl]-2-methylalanine (1 g, 4.21 mmol) in DCM (10 ml anhydrous), cyclohexane (10 ml) at 0° C. under nitrogen was added boron trifluoride diethyl etherate (7 μl, catalytic). tert-Butyl 2,2,2-trichloroacetimidate (1.51 ml, 8.43 mmol) in cyclohexane (10 ml) was then added slowly over 30 minutes before allowing to warm to RT. Reaction was allowed to stir at RT for 16 hours. To the crude reaction mixture was added 190 mg of NaHCO₃ and the reaction filtered. The mother liquors were concentrated in vacuo. The crude extract was purified by column chromatography (10% EtOAc heptane) to yield the desired product (0.863 g, 70%).

¹H NMR (300 MHz, CDCl₃) δ: 7.39-7.31 (5H, m), 5.46 (1H, br s), 5.10 (2H, s), 1.54 (6H, s), 1.45 (9H, s).

Stage 2—t-butyl 2-methylalaninate

To a solution of tert-Butyl N-[(benzyloxy)carbonyl]-2-methylalaninate (0.86 mg, 2.90 mmol) in EtOAc (20 ml) was added 100 mg of 10% palladium on carbon catalyst. The mixture was evacuated and stirred under an atmosphere of hydrogen for 18 hrs., filtered through Celite®, washed with EtOAc and concentrated in vacuo. The product was isolated as a yellow oil (0.45 mg, 96%) which contained traces of EtOAc.

¹H NMR (300 MHz, CDCl₃) δ: 1.48 (9H, s), 1.32 (6H, s).

### Intermediate 33 tert-Butyl N-[(6-{(1E)-3-[(1-isobutoxyethoxy)amino]-3-oxoprop-1-en-1-yl}pyridin-3-yl)methyl]-2-methyl-D-alaninate

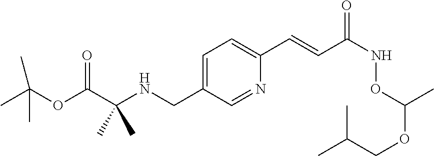

Intermediate 2 (100 mg, 0.34 mmol) and Intermediate 32 (59 mg, 0.37 mmol) in dichloroethane (5 mL) were stirred under nitrogen at room temperature for 20 min. Sodium cyanoborohydride (32 mg, 0.51 mmol) was added and the reaction continued to stir for 6 h. It was then partitioned between dichloromethane (100 mL) and water (100 mL). the organic layer was separated and the aqueous layer extracted with more dichloromethane (50 mL). The combined organic layers were dried (Na₂SO₄) and the solvent removed in vacuo. The residue was purified by column chromatography (silica gel: 0-100% EtOc heptanes) to give the title compound (30 mg) as an orange oil. m/z 436 [M+H]⁺.

### Intermediate 34 3-Methylcyclopentyl N-[(6-{(1E)-3-[(1-isobutoxyethoxy)amino]-3-oxoprop-1-en-1-yl}pyridin-3-yl)methyl]-2-methyl-D-alaninate

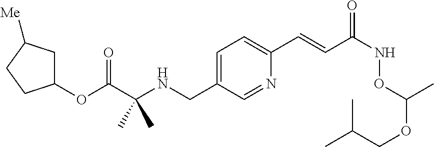

In a similar manner to Intermediate 33 from Intermediate 2 (100 mg, 0.34 mmol). Intermediate 35 (63 mg, 0.34 mmol) and sodium cyanoborohydride (32 mg, 0.51 mmol) to give intermediate 34 (42 mg) as an orange oil. m/z 462 [M+H]⁺.

### Intermediate 35 3-Methylcyclopentyl 2-methylalaninate

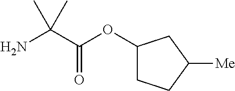

2-Aminoisobutyric acid (1 g, 9.7 mmol), 3-methylcyclopentanol (3.2 mL, 29.1 mmol) and para-toluenesulphonic acid (2.03, 10.67 mmol) were heated to 100° C. in cyclohexane (100 mL) in Dean-Stark apparatus for 72 h. The reaction was then cooled to room temperature. The reaction was filtered and the filtrate concentrated under reduced pressure to a brown oil (1.29 g). The oil was determined to be a 1:1 mixture of Intermediate 35 and 3-methylcyclopentanol by ¹H NMR and was used without further purification.

### EXAMPLES

### Example 1

### Cyclopentyl 1-[({6-[(1E)-3-(hydroxyamino)-3-oxoprop-1-en-1-yl]pyridin-3-yl}methyl)amino]cyclobutanecarboxylate

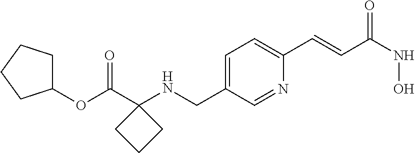 [SMILES: O=C(/C=C/c1ccc(CNC2(C(=O)OC3CCCC3)CCC2)cn1)NO]

Intermediate 5 (0.75 g, 1 eq) and hydroxylamine hydrochloride (0.42 g, 3 eq) were stirred in methanol (8 mL) and cooled to <5° C. Potassium hydroxide (0.68 g, 6 eq) was dissolved in water (2 mL) and charged to the reaction at <5° C. over 5 min. The reaction was stirred for a further 20 min then quenched to pH ˜7 with 4N HCl. Water (20 mL) was charged, aged for 1 h 40 min in an ice bath, then the title compound was isolated by filtration as a solid (0.42 g, 58%), m/z 360 [M+H]^{+ 1}H NMR (300 MHz, d₆-DMSO) δ (ppm): 10.88 (1H, s), 9.10 (1H, s). 8.53 (1H, s), 7.76 (1H, d), 7.49 (2H, dd), 6.90 (1H, d), 5.07 (1H, m), 3.58 (2H, s), 2.87 (1H, bs), 2.27 (2H, m), 2.08-1.48 (12H, m).

The following examples were prepared in manner similar to that of Example 1.

### Example 2

### Cyclopentyl 1-[({6-[(1E)-3-(hydroxyamino)-3-oxoprop-1-en-1-yl]pyridin-3-yl}methyl)amino]cyclohexanecarboxylate

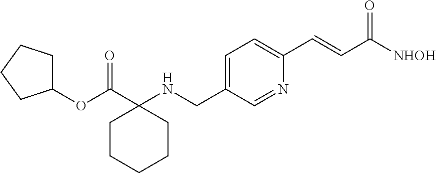

From Intermediate 7 (60 mg, 0.14 mmol)hydroxylamine hydrochloride (40 mg, 0.58 mmol) in the presence of potassium hydroxide (70 mg, 124 mmol) to give the title compound (34 mg). In this case the product was isolated without purification by extraction from the quenched aqueous reaction mixture with ethyl acetate, drying (MgSO₄) and removing the solvent under reduced pressure. m/z 388 [M+H]⁺, ¹H NMR (300 MHz, d₆-DMSO) δ: 10.87 (1H, brs), 9.09 (1H, br s), 8.52 (1H, s), 7.75 (1H, dd), 7.51 (1H, d), 7.45 (1H, d), 6.89 (1H, d), 5.07 (1H, t), 3.58 (2H, s), 2.31-2.50 (2H, m), 1.18-1.89 (16H, m).

### Example 3

### Cyclopentyl 1-({4-[(1E)-3-(hydroxyamino)-3-oxoprop-1-en-1-yl]benzyl}amino)cyclobutanecarboxylate

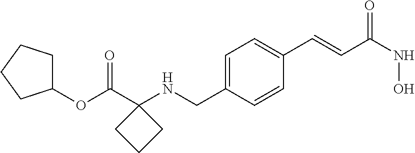 [SMILES: O=C(/C=C/c1ccc(CNC2(C(=O)OC3CCCC3)CCC2)cc1)NO]

From Intermediate 9 (0.63 g, 1.54 mmol) and hydroxylamine hydrochloride (0.32 g, 4.60 mmol) to give the title compound (0.4 g). In this case the title compound was purified by column chromatography [silica gel, ethyl acetate in hexane (25-100%)] after extraction with ethyl acetate from the quenched aqueous reaction mixture. m/z 359 [M+H]^{+ 1}H NMR (300 MHz, d₆-DMSO) δ (ppm): 10.72 (1H, s), 9.02 (1H, s), 7.49 (2H, s), 7.44 (1H, d), 7.36 (2H, d), 6.42 (1H, d), 5.09 (1H, t), 3.55 (2H, s), 2.19-2.21 (2H, m) and 1.55-2.01 (12H, m).

### Example 4

### Cyclopentyl 1-({4-[(1E)-3-(hydroxyamino)-3-oxoprop-1-en-1-yl]benzyl}amino)cyclopentanecarboxylate

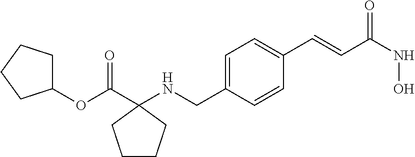 [SMILES: O=C(/C=C/c1ccc(CNC2(C(=O)OC3CCCC3)CCCC2)cc1)NO]

From Intermediate 8 (7.3 g, 17.8 mmol) and hydroxylamine hydrochloride (3.7 g. 53.2 mmol) to give the title compound (3.77 g), m/z 373 [M+H]^{+ 1}H NMR, (300 MHz DMSO) δ (ppm): 10.72 (1H, s), 9.02 (1H, s), 7.51 (2H, d), 7.43 (1H, d), 7.33 (2H, d), 6.43 (1H, d), 5.10 (1H, m), 3.64 (2H, s), 1.99-1.56 (16H, m).

### Example 5

### Cyclopentyl N-{4-[(1E)-3-(hydroxyamino)-3-oxoprop-1-en-1-yl]benzyl}-2-methyl-D-alaninate Trifluoroacetate

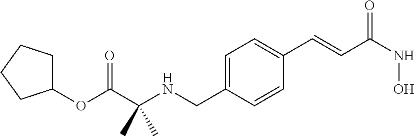

From Intermediate 10 (103.9 mg, 0.30 mmol), hydroxylamine hydrochloride (62.6 mg, 0.90 mmol) and lithium hydroxide (43.2 mg, 1.8 mmol) to give the title compound (3.5 mg) as the trifluoroacetate salt after purification by HPLC. m/z 347 [M+H]^{+ 1}H NMR (300 MHz, CD₃OD) δ (ppm); 7.71 (2H, d), 7.49 (3H, m) 6.61 (1H, m), 5.37 (1H, m) 4.35 (2H, m) 2.00 (2H, m) 1.83-1.54 (12H, m)

### Example 6

### Cyclopentyl 1-({4-[(1E)-3-(hydroxyamino)-3-oxoprop-1-en-1-yl]benzyl}amino)cyclopropanecarboxylate Trifluoroacetate

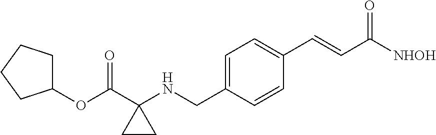

Intermediate 11 (96 mg, 0.22 mmol) was dissolved in dichloromethane/methanol (11 mL, 10:1 v/v) and 4M HCl in dioxane (0.17 mL, 0.66 mmol) was added. After 1 hour the solvent was removed under reduced pressure and the residue purified by HPLC to give the title compound (14.8 mg) as the trifluoroacetate salt. m/z. 345 [M+H]^{+ 1}H NMR (300 MHz, CD₃OD) δ l (ppm); 7.74-7.46 (6H, m), 5.33 (1H, t), 4.44 (2H, 5), 1.96-1.54 (12H, m).

The following compounds were prepared in a similar manner to the compound of Example 6

### Example 7

### Cyclopentyl 1-({4-[(1E)-3-(hydroxyamino)-3-oxoprop-1-en-1-yl]benzyl}amino)cyclohexanecarboxylate Trifluoroacetate

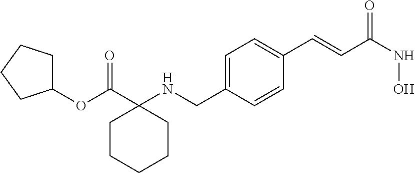 [SMILES: O=C(/C=C/c1ccc(CNC2(C(=O)OC3CCCC3)CCCCC2)cc1)NO]

From Intermediate 12 (64.9 mg, 0.13 mmol) to give the title compound as the trifluoroacetate salt (37 mg). m/z 387 [M+H]^{+ 1}H NMR (300 MHz, CO₃OD) δ (ppm); 7.69 (2H, d), 7.58 (3H, m), 6.54 (1H, m), 5.39 (1H, m), 4.18 (2H, 5), 2.36 (2H, d), 2.02-1.38 (16H, m)

### Example 8

### Cyclopentyl N-{4-[(1E)-3-(hydroxyamino)-3-oxoprop-1-en-1-yl]benzyl}-α-methyl-L-phenylalaninate Trifluoroacetate

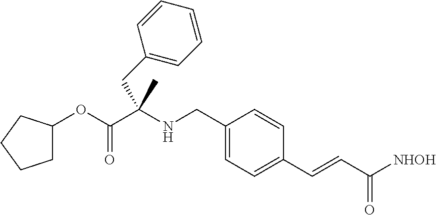

From Intermediate 13 (83 mg, 0.16 mmol) to give the title compound as the trifluoroacetate salt (22.5 mg). m/z 389 [M+H]⁺, ¹H NMR (300 MHZ, CD₃OD) δ (ppm); 7.68 (2H, M), 7.63 (1H, m) 7.57 (2H, M), 7.30 (3H, M), 7.24 (2H, m), 6.56 (1H, d), 5.22 (1H, M), 4.92 (1H, d), 4.14 (1H, d), 3.35 (1H, (1H, d), 3.29 (1H, d), 1.94-1.41 (8H, M), 1.69 (3H, s)

### Example 9

### Cyclopentyl N-{4-[(1E)-3-(hydroxyamino)-3-osoprop-1-en-1-yl]benzyl}-2-methyl-D-leucinate Trifluoroacetate

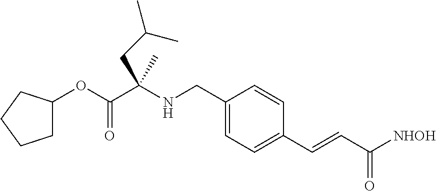

From Intermediate 14 (78.2 mg, 0.16 mmol) to give the title compound (35.7 mg). m/z 389 [M+H]^{+ 1}H NMR (300 MHz, CD₃OD) δ (ppm); 7.67 (3H, M), 7.57 (2H, d), 6.55 (1H, m), 5.36 (1H, m), 4.28 (1H, d), 4.13 (1H, s), 1.98-1.54 (14H, m), 1.00 (6H, s)

### Example 10

### Cyclopentyl N-{4-[(1E)-3-(hydroxyamino)-3-oxoprop-1-en-1-yl]benzyl}-2-methyl-L-leucinate Trifluoroacetate

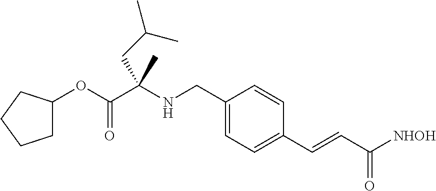

From Intermediate 15 (85 mg, 0.17 mmol) to give the title compound (31.4 mg) as the trifluoroacetate salt. m/z 389 [M+H]^{+ 1}H NMR (300 MHZ, CD₃OD) δ (ppm); 7.67 (2H, d) 7.59 (3H, m), 6.56 (1H, d), 5.36 (1H, m), 4.28 (1H, d), 4.12 (1H, d), 2.03-1.74 (11H, m), 1.71 (3H, 5), 0.98 (6H, m)

### Example 11

### Cyclopentyl N-{4-[(1E)-3-(hydroxyamino)-3-oxoprop-1-en-1-yl]benzyl}-L-isovalinate Trifluoroacetate

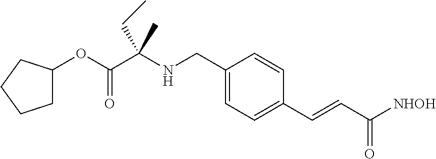

From Intermediate 16 (47 mg) to give the title compound (11.3 mg) as the trifluoroacetate salt. m/z 361 [M+H]⁺, ¹H NMR (300 MHz, CD₃OD) δ (ppm); 7.69 (2H, d), 7.60 (1H, d) 7.56 (2H, d), 6.57 (1H, d), 5.36 (1H, m), 4.30 (1H, d), 4.16 (1H, d), 2.16-1.66 (10H, m), 1.60 (3H, s), 1.05 (3H, t)

### Example 12

### Cyclopentyl N-{4-[(1E)-3-(hydroxyamino)-3-oxoprop-1-en-1-yl]benzyl}-3-methyl-L-isovalinate Trifluoroacetate

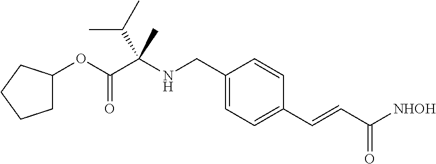

From Intermediate 17 (75.7 mg, 0.16 mmol) to give the title compound (17 mg) as the trifluoroacetate salt. m/z 375 [M+H]^{+ 1}H NMR (300 MHz, CD₃OD) δ (ppm); 7.68 (2H, d), 7.60 (1H, d), 7.58 (2H, d), 6.57 (1H, d), 5.35 (1H, m), 4.38 (1H, d), 4.12 (1H, d), 2.38 (1H, septet) 1.84-1.69 (8H, m), 1.63 (3H, d), 1.13 (3H, d), 1.05 (3H, d)

### Example 13

### Cyclopentyl 1-[({6-[(1E)-3-(hydroxyamino)-3-oxoprop-1-en-1-yl]pyridin-3-yl}methyl)amino]cyclopentanecarboxylate Trifluoroacetate

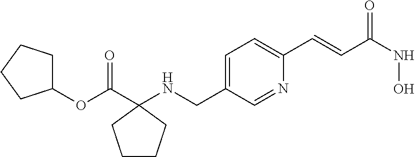 [SMILES: O=C(/C=C/c1ccc(CNC2(C(=O)OC3CCCC3)CCCC2)cn1)NO]

From Intermediate 6 (84.7 mg, 0.18 mmol) to give the title compound (34.9 mg) as the trifluoroacetate salt. m/z 374 [M+H]^{+ 1}H NMR (300 MHz, CD₃OD) δ (ppm); 8.75 (1H, s), 8.06 (1H, d), 7.70 (1H, d), 7.60 (1H, d), 6.97 (1H, d), 5.39 (1H, m), 4.35 (2H, s), 2.38 (2H, m), 2.13 (2H, m), 1.97 (6H, m), 1.77 (6H, m).

### Example 14

### Cyclopentyl 1-({4-[3-hydroxyamino)-3-oxopropyl]benzyl}amino) cyclopentanecarboxylate

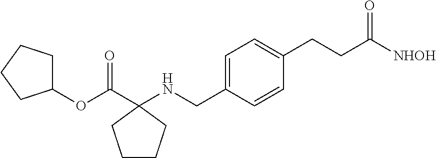

To a solution of Intermediate 4 (208 mg, 0.68 mmol) and Intermediate 19 (184 mg, 0.68 mmol) in dichloromethane (20 mL) was added sodium triacetoxyborohydride (430 mg, 2.04 mmol) and acetic acid (47 μL). The resulting solution was stirred at room temperature for 5 h and then quenched with saturated NH₄Cl. The reaction was extracted with dichloromethane (2×50 mL) and the combined organic layers were dried (MgSO₄) and concentrated in vacuo. The resulting residue was dissolved in 4M HCl dioxane (5 mL) and stirred at Mom temperature for 1 h. The reaction was quenched with satd. NaHCO₃ and extracted with ethyl acetate (2×150 mL). The combined organic layers were dried (MgSO₄) and evaporated. The residue was purified by HPLC to give the title compound (80 mg) as a colourless solid. m/z 375 [M+H]^{+ 1}H NMR (300 MHz, CD₃OD), δ (ppm): 7.48 (2H, d J=7.9 Hz), 7.36 (2H, d J=8.1 Hz), 5.40 (1H, m), 4.18 (2H, s), 2.98 (2H, t, J=7.2), 2.38 (4H, m), 2.08-1.52 (14H, m)

The following examples were made in a similar manner to the title compound of Example 14.

### Example 15

### Cyclopentyl N-{4-[3-(hydroxyamino)-3-oxopropyl]benzyl}-2-methyl-D-alaninate

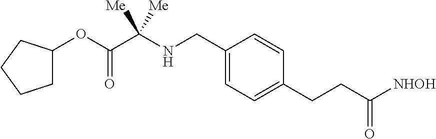

From Intermediate 4 (140 mg, 0.47 mmol) and Intermediate 21 (89 mg, 0.52 mmol) to give the title compound (130 mg) as a colourless solid. m/z 349 [M+H]⁺. ¹H NMR (300 MHz, CD₃OD), δ (ppm): 7.47 (2H, d J=8.1 Hz), 7.35 (2H, d J=81 Hz), 5.38-5.34 (1H, m), 4.18 (2H, s), 2.98 (2H, t J=7.5), 2.41 (2H, t J=7.5 Hz), 1.98-1.66 (8H, m), 1.60 (6H, s)

### Example 16

### Cyclopentyl N-{4-[3-(hydroxyamino)-3-oxopropyl]benzyl}-2-methyl-D,L-leucinate

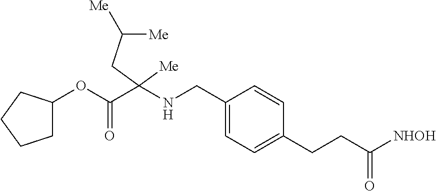

From Intermediate 4 (209 mg, 0.68 mmol) and Intermediate 21 (146 mg, 0.61 mmol) to give the title compound (200 mg) as a colourless solid, m/z 391.51 [M+H]⁺

### Example 17

### Cyclopentyl N-{4-[3-(hydroxyamino)-3-oxopropyl]benzyl}-3-methyl-L-isovalinate

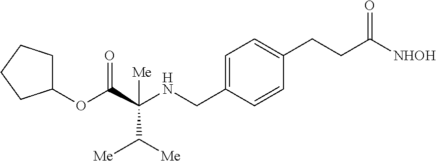

From Intermediate 4 (200 g, 0.68 mmol) and Intermediate 22 (140 mg, 0.68 mmol) to give the title compound (21 mg) as a colourless solid, m/z 377 [M+H]⁺. ¹H NMR (300 MHz, CD₃OD) δ (ppm): 7.43-7.48 (2H, m), 7.32-7.37 (2H, m), 5.35 (1H, td, J=5.4, 2.9 Hz), 4.02-4.33 (2H, m), 2.97 (2H, t, J=7.5 Hz), 2.41 (2H, t, J=7.4 Hz), 1.67-2.05 (8H, m), 1.60 (3H, s), 1.02-1.15 (6H, m)

### Example 18

### 1-({4-[3-(Hydroxyamino)-3-oxopropyl]benzyl}amino)cyclo pentanecarboxylic acid

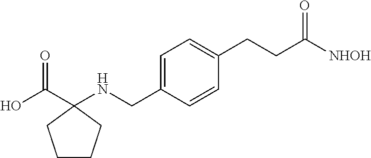

To a solution of Intermediate 4 (208 mg, 68 mmol) and intermediate 19 (184 mg, 0.68 mmol) in dichloromethane (20 mL) was added sodium triacetoxyborohydride (430 mg, 204 mmol) and acetic acid (47 μL). The resulting solution was stirred at room temperature for 5 h and then quenched with saturated NH₄Cl. The reaction was extracted with dichloromethane (2×50 mL) and the combined organic layers were dried (MgSO₄) and concentrated in vacuo. The solid residue (40 mg) was stirred with lithium hydroxide (40 mg, 15 mmol) in THF (1 mL) and water (1 mL) at 45° C. for 36 h. The reaction was concentrated under reduced pressure and the resulting residue purified by prep HPLC. The purified carboxylic acid derivative was stirred in dichloromethane-TFA (1 mL, 1:1 v/v) for 1 h at room temperature and the reaction concentrated under reduced pressure. The residue was subjected to prep HPLC to give the title compound (3 mg) as a colourless solid. ¹H NMR (300 MHz, CD₃OD), δ (ppm): 7.47 (2H, d J=7.9 Hz), 7.36 (2H, d J=8.1 Hz), 4.18 (2H, s), 3.01-2.95 (2H, t J=7.5), 2.38 (4H, m), 1.97-1.59 (6H, m)

### Example 19

### N-(4-[3-(Hydroxyamino)-3-oxopropyl]benzyl)-2-methyl-D,L-leucine

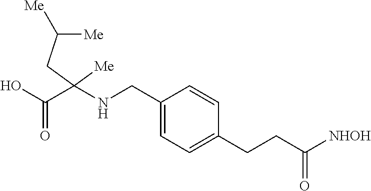

To a solution of Intermediate 4 (150 mg, 51 mmol) and (R,S)-α-methylleucine (75 mg, 51 mmol) DCE (20 mL) was added sodium triacetoxyborohydride (323 mg, 183 mmol) and the reaction was stirred at room temperature for 12 h. The reaction was concentrated under reduced pressure and the resulting residue purified by HPLC to give the title compound (1 mg) as a colourless solid. m/z 332 [M+H]^{+ 1}H NMR (300 MHz, CD₃OD), δ (ppm): 7.44 (2H, d J=8.1 Hz), 7.35 (2H, d J=8.1 Hz), 4.20 (1H, d J=12.4 Hz), 4.09 (1H, d, J=12.6 Hz), 2.97 (2H, t J=7.3 Hz), 2.41 (2H, t J=7.7 Hz), 2.01-1.87 (3H, m), 1.76 (3H, s), 1.01-6H, t J=6.3 Hz).

### Example 20

### 1-({4-[(1E)-3-(Hydroxyamino)-3-oxoprop-1-en-1-yl]benzyl}amino) cyclopentanecarboxylic acid Trifluoroacetate

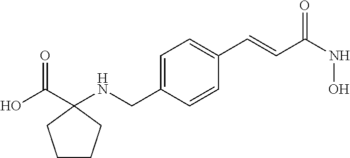 [SMILES: O=C(/C=C/c1ccc(CNC2(C(=O)O)CCCC2)cc1)NO]

Intermediate 30 (13.6 mg, 0.034 mmol) was dissolved in CH₂Cl₂/MeOH [10:1 v/v] (11 mL), 4M HCl (0.042 mL, 0.168 mmol) was added and the reaction stirred at room temperature for 1 h. The solvent was removed in vacuo and the residue purified by HPLC to give the title compound (2.4 mg). m/Z 305 [M+H]^{+ 1}H NMR (300 MHz, CD₃OD) δ (ppm); 7.67-7.59 (6H, m), 4.25 (2H, 5), 2.41 (2H, cl), 2:19-1.86 (6H, rule

### Example 2

### 1-[({6-[(1E)-3-(hydroxyamino)-3-oxoprop-1-en-1-yl]pyridin-3-yl}methyl)amino]cyclopentanecarboxylic acid Trifluoroacetate

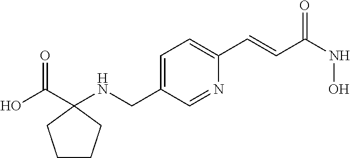 [SMILES: O=C(/C=C/c1ccc(CNC2(C(=O)O)CCCC2)cn1)NO]

In a manner similar to the method for Example 20 from Intermediate 31 (70 mg, 0.17 mmol) to give the title compound (63 mg) as the trifluoroacetate salt after purification by HPLC m/z 306 [M+H]^{+ 1}H NMR (300 MHz, CD₃OD) δ (ppm); 8.78 (1H, s), 8.12 (1H, d), 7.75 (1H, d), 7.59 (1H, d), 6.98 (1H, d) 4.37 (2H, s), 2.43 (2H, m), 2.17 (2H, m), 1.97 (4H, m)

### Example 22

### 1-[({6-[(1E)-3-(Hydroxyamino)-3-oxoprop-1-en-1-yl]pyridin-3-yl}methyl)amino]cyclobutanecarboxylic acid

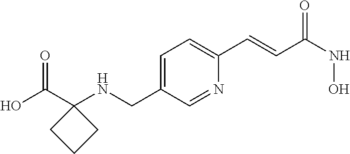 [SMILES: O=C(/C=C/c1ccc(CNC2(C(=O)O)CCC2)cn1)NO]

The compound of Example 1 (0.1 g, 0.27 mmol) was stirred with 1N NaOH (10 mL) in methanol (10 mL) for 19 h. The reaction was acidified to pH7 with 4N HCl and the resulting solid was collected by filtration and washed with water and EtOAc, and then dried to give the title compound (52.7 mg). m/z 292 [M+H]⁺¹H NMR (300 MHz, d₆-DMSO) δ (ppm): 10.89 (1H, s), 8.57 (1H, s), 7.81 (1H, d), 7.54 (1H, d), 7.46 (1H, d), 6.92 (1H, d), 3.68 (2H, s), 2.30-1.69 (6H, m).

### Example 23

### t-Butyl 1-[({6-[(1E)-3-(hydroxyamino)-3-oxoprop-1-en-1-yl]pyridin-3-yl}methyl)amino]-2-methyl-D-alaninate Trifluoroacetate

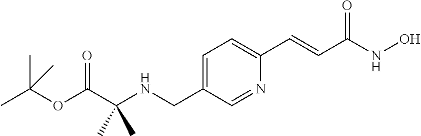

Following the procedure of Example 6, from Intermediate 33 (30 mg, 0.069 mmol) to give the title compound (7.7 mg) after purification by HPLC. m/z 336 [M+H]⁺, ¹H NMR (300 MHz, DMSO-d6) δ ppm; 9.46 (1H, bs), 8.69 (1H, m), 7.95 (1H, m), 7.88 (1H, m), 7.69 (1H, m), 7.52 (1H, d, J=15.6 Hz), 7.08 (1H, 5), 6.97 (1H, cl, J=15.6 Hz), 4.21 (2H, m), 1.57 (6H, s), 1.51 (9H, s).

### Example 24

### 3-Methylcyclopentyl N-({6-[(1E)-3-(hydroxyamino)-3-oxoprop-1-en-1-yl]pyridin-3-yl}methyl)-2-methyl-L-alaninate Trifluoroacetate

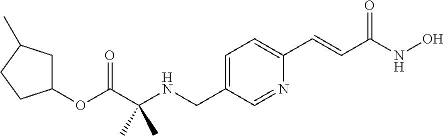

Following the procedure of Example 6 from intermediate 34 (42 mg, 0.091 mmol) to give the title compound (2.4 mg) after purification by HPLC as a white solid and a mixture of diastereoisomers. m/z 362 [M+H]^{+ 1}H NMR (300 MHz, DMSO-d6) δ ppm; 10.98 (1H, s), 9.58 (2H, m), 8.67 (1H, m), 7.93 (1H, m), 7.69 (1H, d, J=7.8 Hz), 7.51 (1H, m), 7.01 (1H, m), 5.19 (1H, m), 4.22 (2H, m), 2.30-1.80 (5H, m), 1.58 (6H, m), 1.25 (2H, m), 1.02 (3H, m).

### Measurement of Biological Activities

### Histone Deacetylase Activity

The ability of compounds to inhibit histone deacetylase activities was measured using the commercially available HDAC fluorescent activity assay from Biomol. in brief, the Fluor de Lys™ substrate, a lysine with an epsilon-amino acetylation, is incubated with the source of histone deacetylase activity (HeLa nuclear extract) in the presence or absence of inhibitor. Deacetylation of the substrate sensitises the substrate to Fluor de Lys™ developer, which generates a fluorophore. Thus, incubation of the substrate with a source of HDAC activity results in an increase in signal that is diminished in the presence of an HDAC inhibitor.

Data are expressed as a percentage of the control, measured in the absence of inhibitor, with background signal being subtracted from all samples, as follows:

% activity=[(Sⁱ−B)/(S^{o}−B)]×100

where Sⁱ is the signal in the presence of substrate, enzyme and inhibitor, S^{o} is the signal in the presence of substrate, enzyme and the vehicle in which the inhibitor is dissolved, and B is the background signal measured in the absence of enzyme.

Histone deacetylase activity from crude nuclear extract derived from HeLa celLs was used for screening. The preparation, purchased from Cilbiotech (Mons, Belgium), was prepared from HeLa cells harvested whilst in exponential growth phase. The nuclear extract was prepared according to the methodology described by J. D. Dignam et al, Nucl. Acid. Res., 1983, 11, 1475-1489. The final buffer composition was 20 mM HEPES pH7.9, 100 mM KCl, 0.2 mM EDTA, 0.5 mM DTT, 0.5 mM PMSF and 20% (v/v) glycerol.

Dose response curves were generated from 8 compound concentrations (top concentration 10 μM, with 3-fold dilutions), using duplicate points.

IC₅₀ results were allocated to one of 3 ranges as follows:

Range A: IC₅₀<100 nM; Range B: IC₅₀ from 101 nM to 1000 nM; Range C: IC₅₀>1001 nM

NT=Not tested

### U937 and HUT Cell Inhibition Assay

Cancer cell lines (U937 and HUT) growing in log phase were harvested and seeded at 1000-2000 cells/well (100 μl final volume) into 96-well tissue culture plates. Following 24 h of growth cells were treated with Compound. Plates were then re-incubated for a further 72-98 h before a WST-1 cell viability assay was conducted according to the suppliers (Roche Applied Science) instructions.

Data were expressed as a percentage inhibition of the control, measured in the absence of inhibitor, as follows:

% inhibition=100−[(Sⁱ/S^{o})×100]

where Sⁱ is the signal in the presence of inhibitor and S^{o} is the signal in the presence of DMSO.

Dose response curves were generated from 8 concentrations (top final concentration 10 μM, with 3-fold dilutions), using 6 replicates.

IC₅₀ values were determined by non-linear regression analysis, after fitting the results to the equation for sigmoidal dose response with variable slope (% activity against log concentration of Compound), using Graphpad Prism software.

IC₅₀ results were allocated to one of 3 ranges as follows:

Range A: IC₅₀<100 nM; Range B: IC₅₀ from 101 nM to 1000 nM; Range C: IC₅₀>1000 nM NT=Not tested

### LPS-Stimulation of Human Whole Blood

Whole blood was taken by venous puncture using heparinised vacutainers (Becton Dickinson) and diluted in an equal volume of RPMI1640 tissue culture media (Sigma). 100 μl was plated in V-bottomed 96 well tissue culture treated plates. 2 hrs after the addition of the inhibitor in 100 μl of RPMI 1640 media, the blood was stimulated with LPS (E coli strain 005:B5, Sigma) at a final concentration of 100 ng/ml and incubated at 37° C. in 5% CO₂ for 6 hrs. TNF-α levels were measured from cell-free supernatants by sandwich ELISA (R&D Systems #QTA00B)

IC50 values were allocated to one of three ranges as follows:

Range A: IC50<100 nM

Range B: IC₅₀ from 101 nM to 1000 nM

Range C: IC50>1000 nM

NT=Not tested

TABLE 1
Results

| | | | | Inhibitor |
|---|---|---|---|---|
| | Inhibitor | Inhibitor | Inhibitor | activity versus |
| | activity | activity | activity | human whole |
| | versus | versus U937 | versus Hut78 | blood |
| Example | HDAC | proliferation | proliferation | TMFα release |
| 1 | B | B | B | A |
| 2 | B | B | C | A |
| 3 | B | B | C | A |
| 4 | B | B | C | A |
| 5 | C | B | C | A |
| 6 | C | C | C | B |
| 7 | C | B | C | B |
| 8 | B | A | C | A |
| 9 | C | B | C | B |
| 10 | B | B | C | A |
| 11 | B | A | C | A |
| 12 | C | C | C | B |
| 13 | B | A | C | A |
| 14 | C | B | C | A |
| 15 | C | B | C | A |
| 16 | C | B | C | B |
| 17 | C | B | C | NT |
| 18 | C | NT | NT | NT |
| 19 | C | NT | NT | NT |
| 20 | B | NT | NT | NT |
| 21 | B | NT | NT | NT |
| 22 | B | NT | NT | NT |
| 23 | B | C | C | B |
| 24 | B | B | C | NT |

Broken Cell Carboxyleste rase Assay

Any given compound of the present invention wherein R₁ is an ester group may be tested to determine whether it meets the requirement that it be hydrolysed by intracellular esterases, by testing in the following assay.

### Preparation of Cell Extract

U937 or Hut78 tumour cells (˜10⁹) were washed in 4 volumes of Dulbeccos PBS (˜litre) and pelleted at 525 g for 10 min at 4° C. This was repeated twice and the final cell pellet was resuspended in 35 ml of cold homogenising buffer (Trizma 10 mM, NaCl 130 mM, CaCl₂ 0.5 mM pH 7.0 at 25° C.). Homogenates were prepared by nitrogen cavitation (700 psi for 50 min at 4° C.). The homogenate was kept on ice and supplemented with a cocktail of inhibitors at final concentrations of:

Leupeptin 1 μM

Aprotinin 0.1 μM

E64 8 μM

Pepstatin 1.5 μM

Bestatin 162 μW

Chymostatin 33 μM

After clarification of the cell homogenate by centrifugation at 525 g for 10 min, the resulting supernatant was used as a source of esterase activity and was stored at −80° C. until required.

### Measurement of Ester Cleavage

Hydrolysis of esters to the corresponding carboxylic acids can be measured using the cell extract, prepared as above. To this effect cell extract (˜30 μg/total assay volume of 0.5 ml) was incubated at 37° C. in a Tris- HCl 25 mM, 125 mM NaCl buffer, pH 7.5 at 25° C. At zero time the ester (substrate) was then added at a final concentration of 2.5 μM and the samples were incubated at 37° C. for the appropriate time (usually 0 or 80 min). Reactions were stopped by the addition of 3× volumes of acetonitrile. For zero time samples the acetonitrile was added prior to the ester compound. After centrifugation at 12000 g for 5 min, samples were analysed for the ester and its corresponding carboxylic acid at room temperature by LCMS (Sciex API 3000, HP1100 binary pump, CTC PAL). Chromatography was based on an AcCN (75×2.1 mm) column and a mobile phase of 5-95% acetonitrile in water/0.1% formic acid. Rates of hydrolysis are expressed in pg/mL/min.

### HCE-1 Carboxylesterase Assay

Hydrolysis of esters to the corresponding carboxylic acids by hCE-1 can be measured using the following procedure. At zero time, 100 μl of recombinant hCE-1 at a concentration of 6 μg/ml in phosphate assay buffer (K₂PO₄100 mM, KCl 40 mM, PH 7.4) was added to an equal volume of assay buffer containing 5 μM ester substrate. After thorough mixing, triplicate samples were incubated for 0, 20 or 80 minutes at 37° C. At the appropriate time, hydrolysis was stopped by the addition of 600 μl of acetonitrile. For zero time samples, the acetonitrile was added prior to the enzyme. The samples were analysed for the ester and its corresponding carboxylic acid at room temperature by LCMS (Sciex API 3000, HP1100 binary pump, CTC PAL), Chromatography was based on an AcCN (75×2.1 mm) column and a mobile phase of 5-95% acetonitrile in water/0.1% formic acid. Levels of the acid, the hydrolysis product, after 80 minutes are expressed ng/ml.

| | | | Inhibition of | Inhibition of | |
|---|---|---|---|---|---|
| | Inhibition | | proliferation | proliferation in | Ratio IC50s |
| | of HDAC | | in U937 cells | HUT78 cells | in HUT 78 |
| Compound | (IC50 nM) | | (IC50 nM) | (IC50 nM) | to U937 cells |
| 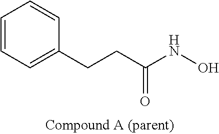 [SMILES: O=C(CCc1ccccc1)NO] | 2600 | | 13% @ 10 μM | 10% @ 10 μM | ~1 |
| 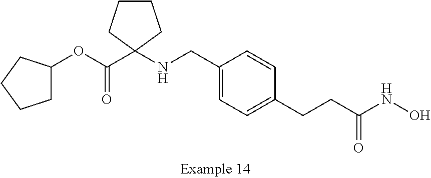 [SMILES: O=C(CCc1ccc(CNC2(C(=O)OC3CCCC3)CCCC2)cc1)NO] | Ester 4200 | Acid 6120 | 180 | 6200 | 34 |
| 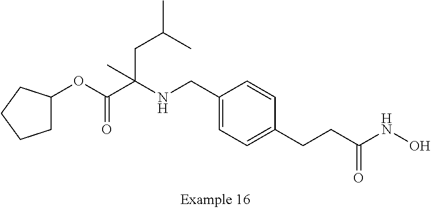 [SMILES: CC(C)CC(C)(NCc1ccc(CCC(=O)NO)cc1)C(=O)OC1CCCC1] | 4900 | 6242 | 190 | 0.04 | ND |

Table 2 shows that the acid of examples 14 and 16 have similar IC50s in the above enzyme assay to the parent compound A, indicating that binding to the enzyme has not been disrupted by attachment of the esterase motif. Di-substituted compounds are hydrolysed by hCE-1 in the above assay and as a consequence the acid accumulates in cells. This accumulation of acid results in Examples 14 and 16 being significantly more potent than the parent compound in the U937 cellular assay above. These data highlight the potency benefit that can be achieved by the attachment of the esterase motif.

TABLE 3

| | | | Inhibition of | Inhibition of | |
|---|---|---|---|---|---|
| | Inhibition | | proliferation | proliferation | Ratio IC50s |
| | of HDAC | | in U937 cells | in HUT78 cells | in HUT 78 |
| Compound | (IC50 nM) | | (IC50 nM) | (IC50 nM) | to U937 cells |
| 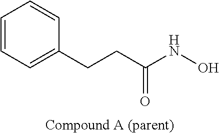 [SMILES: O=C(CCc1ccccc1)NO] | 2600 | | 13% @ 10 μM | 10% @ 10 μM | ~1 |
| 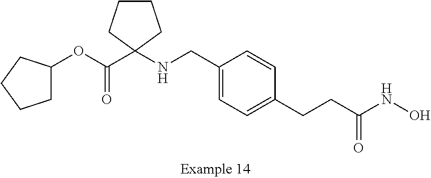 [SMILES: O=C(CCc1ccc(CNC2(C(=O)OC3CCCC3)CCCC2)cc1)NO] | Ester 4200 | Acid 6120 | 180 | 6200 | 34 |
| 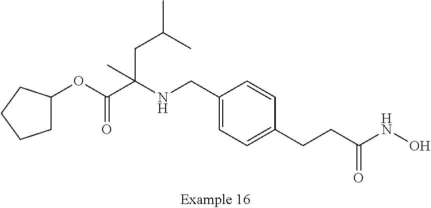 [SMILES: CC(C)CC(C)(NCc1ccc(CCC(=O)NO)cc1)C(=O)OC1CCCC1] | 4900 | 6242 | 190 | 5500 | 30 |

Table 3 shows that the parent compound A has similar potencies in monocytic (U937) and non monocytic (Hut78) cell lines whereas Examples 14 and 16 are 30 times more potent in the monocytic cell line than the non-monocytic cell line. These data highlight the macrophage selectivity of the compounds.

1.-28. (canceled)

29. A compound of formula (I) or a pharmaceutically acceptable salt thereof: 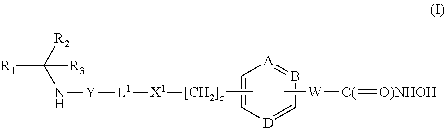
wherein
one of A, B and D is ═N— and the others are each ═CH—;
W is CH═CH— or CH₂CH₂—;
R₁ is a carboxylic acid group (—COOH), or an ester group which is hydrolysable by one or more intracellular carboxylesterase enzymes to a carboxylic acid group wherein the ester group is an ester group of formula R₁₂OC(═O)— wherein R₁₂ is R₇R₅CR₉— wherein
(i) R₇ is hydrogen or optionally substituted (C₁-C₃)alkyl-(Z¹)ₐ-[(C₁-C₃)alkyl]_{b}— or (C₂-C₃)alkenyl-(Z¹)ₐ-[(C₁-C₃)alkyl]_{b}— wherein a and b are independently 0 or 1 and Z¹ is —O—, —S—, or —NR₁₃— wherein R₁₃ is hydrogen or (C₁-C₃)alkyl; and R₈ and R₉ are independently hydrogen or (C₁-C₃)alkyl-; or
(ii) R₇ is hydrogen or optionally substituted R₁₄R₁₅N—(C₁-C₃)alkyl- wherein R₁₄ is hydrogen or (C₁-C₃)alkyl and R₁₅ is hydrogen or (C₁-C₃)alkyl; or R₁₄ and R₁₅ together with the nitrogen to which they are attached form an optionally substituted monocyclic heterocyclic ring of 5- or 6- ring atoms or bicyclic heterocyclic ring system of 8 to 10 ring atoms, and R₈ and R₉ are independently hydrogen or (C₁-C₃)alkyl-; or
(iii) R₇ and R₈ taken together with the carbon to which they are attached form an optionally substituted monocyclic carbocyclic ring of from 3 to 7 ring atoms or bicyclic carbocyclic ring system of 8 to 10 ring atoms, and R₉ is hydrogen,
and wherein in cases (i), (ii) and (iii), “alkyl” includes fluoroalkyl;
one of R₂ and R₃ is a C₁-C₆ alkyl substituent, and the other is methyl, ethyl, n- or iso-propyl, n-, sec- or tert-butyl, phenyl, benzyl, thienyl, cyclohexyl, or cyclohexylmethyl, or R₂ and R₃, taken together with the carbon to which they are attached, form a 3-6 membered saturated cycloalkyl or heterocyclyl ring;
Y is a bond, —C(═O)—, —S(═O)₂—, —C(═O)O—, —C(═O)NR′—, —C(═S)—NR′, —C(═NH)NR′ or —S(═O)₂NR′— wherein R′ is hydrogen or optionally substituted C₁-C₆ alkyl;
L¹ is a divalent radical of formula (Alk¹)ₘ(Alk²)ₚ— wherein
m and p are independently 0 or 1,
Alk¹ and Alk² independently represent optionally substituted divalent C₃-C₇ cycloalkyl radicals, or optionally substituted straight or branched, C₁-C₆ alkylene, C₂-C₆ alkenylene, or C₂-C₆ alkynylene radicals which may optionally contain or terminate in an ether (—O—), thioether (—S—) or amino (—NR^{A}—) link wherein R^{A} is hydrogen or optionally substituted C₁-C₃ alkyl;
X¹ represents a bond; —C(═O); or —S(═O)₂—; NR₄C(═O)—, —C(═O)NR₄—, —NR₄C(═O)NR₅—, —NR₄S(═O)₂—, or —S(═O)₂NR₄— wherein R₄ and R₅ are independently hydrogen or optionally substituted C₁-C₆ alkyl; and
z is 0 or 1.

30. A compound as claimed in claim 29 wherein, unless otherwise specified, the term “substituted” means substituted with up to four substituents selected from (C₁-C₆)alkyl, (C₁-C₆)alkoxy, hydroxy, hydroxy(C₁-C₆)alkyl, mercapto, mercapto(C₁-C₆)alkyl, (C₁-C₆)alkylthio, phenyl, halo (including fluoro, bromo and chloro), trifluoromethyl, trifluoromethoxy, nitro, nitrile (—CN), oxo, —COOH, —COOR^{A}, —COR^{A}, —SO₂R^{A}, —CONH₂, —SO₂NH₂, —CONHR^{A}, —SO₂NHR^{A}, —CONR^{A}R^{B}, —SO₂NR^{A}R^{B}, —NH₂, —NHR^{A}, —NR^{A}R^{B}, —OCONH₂, —OCONHR^{A}, —OCONR^{A}R^{B}, —NHCOR^{A}, —NHCOOR^{A}, —NR^{B}COOR^{A}, —NHSO₂OR^{A}, —NR^{B}SO₂OH, —NR^{B}SO₂OR^{A}, —NHCONH₂, —NR^{A}CONH₂, —NHCONHR^{B}—NR^{A}CONHR^{B}, —NHCONR^{A}R^{B} and —NR^{A}CONR^{A}R^{B} wherein R^{A} and R^{B} are independently a (C₁-C₆)alkyl, (C₃-C₆) cycloalkyl, phenyl or monocyclic heteroaryl having 5 or 6 ring atoms, or R^{A} and R^{B} when attached to the same nitrogen atom form a cyclic amino group.

31. A compound as claimed in claim 29 wherein the radical HONHC(═O)—W— is attached to the ring containing A, B and C in a position meta- or para- to the radical R₂R₃C—NHYL¹X¹[CH₂]_{z}—.

32. A compound as claimed in claim 29 wherein, in the radical R₁R₂R₃C—NHYL¹X¹[CH₂]_{z}—, z is 0.

33. A compound as claimed in claim 29 wherein, in the radical R₁R₂R₃C—NHYL¹X¹[CH₂]_{z}—, Y is a bond.

34. A compound as claimed in claim 29 wherein, in the radical R₁R₂R₃C—NHYL¹X¹[CH₂]_{z}—, X¹ is a bond.

35. A compound as claimed in claim 29 wherein, in the radical R₁R₂R₃C—NHYL¹X¹[CH₂]_{z}—, z is 0, Y and X¹ are each a bond, and L¹ is a divalent radical of formula -(Alk¹)ₘ(Alk²)ₚ— wherein one of m and p is 0 and the other is 1.

36. A compound as claimed in claim 29 wherein the radical —YL¹X¹[CH₂]_{z}— is —CH₂—.

37. A compound as claimed in claim 29 wherein Rᵢ is an ester group of formula R₁₂OC(═O)— wherein R₁₂ is methyl, trifluoromethyl, ethyl, n- or iso-propyl, n-, sec- or tert-butyl, cyclopentyl, methyl-substituted cyclopentyl, cyclohexyl, allyl, bicyclo[2.2.1]hept-2-yl, 2,3-dihydro-1H-inden-2-yl, phenyl, benzyl, 2-, 3- or 4-pyridylmethyl, N-methylpiperidin-4-yl, tetrahydrofuran-3-yl or methoxyethyl.

38. A compound as claimed in claim 29 wherein R₁ is an ester group of formula R₁₂OC(═O)— wherein R₁₂ is cyclopentyl.

39. A compound as claimed in claim 29 wherein one of the substitutents R₂ and R₃ is a C₁-C₆ alkyl substituent, and the other is selected from the group consisting of methyl, ethyl, n- and iso-propyl, n-, sec- and tert-butyl, phenyl, benzyl, thienyl, cyclohexyl, and cyclohexylmethyl; or R₂ and R₃ taken together with the carbon to which they are attached form a 3-6 membered saturated cycloalkyl ring.

40. A compound as claimed in claim 29 wherein one of R₂ and R₃ is methyl or ethyl, and the other is benzyl or C₁-C₆ alkyl; or R₂ and R₃ taken together with the carbon to which they are attached form a cyclopropyl, cyclobutyl, cyclopentyl or cyclohexyl ring.

41. A compound as claimed in claim 29 wherein one of R₂ and R₃ is methyl and the other is methyl, ethyl, n- or iso-propyl, benzyl or n, sec or tert butyl; or R₂ and R₃ taken together with the carbon to which they are attached form a cyclopropyl, cyclobutyl, cyclopentyl or cyclohexyl ring.

42. A compound as claimed in claim 29 having formula (IE): 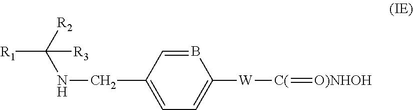
wherein R₁, W and B are as defined in claim 1, one of R₂ and R₃ is methyl, and the other is methyl, ethyl, n- or iso-propyl, benzyl or n, sec or tert butyl; or R₂ and R₃ taken together with the carbon to which they are attached form a cyclopropyl, cyclobutyl, cyclopentyl or cyclohexyl ring.

43. A compound as claimed in claim 42 wherein R₁ is an ester group of formula R₁₂OC(═O)— wherein R₁₂ is cyclopentyl.

44. A compound as claimed in claim 42 wherein W is CH═CH—.

45. A compound as claimed in claim 29 which is in pharmaceutically acceptable salt form.

46. A compound as claimed in claim 29 selected from the group consisting of:
Cyclopentyl 1-[({6-[(1E)-3-(hydroxyamino)-3-oxoprop-1-en-1-yl]pyridin-3-yl}methyl)amino]cyclobutanecarboxylate,
Cyclopentyl 1-[({6-[(1E)-3-(hydroxyamino)-3-oxoprop-1-en-1-yl]pyridin-3-yl}methyl)amino]cyclohexanecarboxylate,
Cyclopentyl 1-[({6-[(1E)-3-(hydroxyamino)-3-oxoprop-1-en-1-yl]pyridin-3-yl}methyl)amino]cyclopentanecarboxylate,
1-[({6-[(1E)-3-(hydroxyamino)-3-oxoprop-1-en-1-yl]pyridin-3-yl}methyl)amino]cyclopentanecarboxylic acid,
1-[({6-[(1E)-3-(Hydroxyamino)-3-oxoprop-1-en-1-yl]pyridin-3-yl}methyl)amino]cyclobutanecarboxylic acid,
t-Butyl 1-[({6-[(1E)-3-(hydroxyamino)-3-oxoprop-1-en-1-yl]pyridin-3-yl}methyl)amino]-2-methyl-D-alaninate,
3-Methylcyclopentyl N-({6-[(1E)-3-(hydroxyamino)-3-oxoprop-1-en-1-yl]pyridin-3-yl}methyl)-2-methyl-L-alaninate,
or a pharmaceutically acceptable salt thereof.

47. A compound as claimed in claim 29 selected from the group consisting of:
Cyclopentyl 1-[({6-[(1E)-3-(hydroxyamino)-3-oxoprop-1-en-1-yl]pyridin-3-yl}methyl)amino]cyclobutanecarboxylate,
Cyclopentyl 1-[({6-[(1E)-3-(hydroxyamino)-3-oxoprop-1-en-1-yl]pyridin-3-yl}methyl)amino]cyclohexanecarboxylate,
or a pharmaceutically acceptable salt thereof.

48. A pharmaceutical composition comprising a compound as claimed in claim 29 wherein R₁ is an ester group as defined in claim 29, together with a pharmaceutically acceptable carrier.

49. A method for the treatment of cell-proliferation disease, polyglutamine disease, neurodegenerative disease, autoimmune disease, inflammatory disease, organ transplant rejection, diabetes, haematological disorders or inflammatory sequelia of infection which comprises administering to a subject suffering from such disease an effective amount of a compound as claimed in claim 1 wherein R₁ is an ester group as defined in claim 29.

50. The method as claimed in claim 49 wherein the treatment is of cancer cell proliferation.

51. The method as claimed in claim 49 wherein the treatment is of rheumatoid arthritis.

52. A method for the treatment of a disease which responds to inhibition of HDAC activity, which comprises administering to a subject suffering such disease an effective amount of a compound as claimed in claim 29 wherein R₁ is an ester group as defined in claim 1.

53. A method as claimed in claim 52 wherein the disease is transplant rejection, rheumatoid arthritis, psoriatic arthritis, Type 1 diabetes, asthma, inflammatory bowel disease, systemic lupus erythematosis, and inflammation accompanying infectious conditions (e.g., sepsis), psoriasis, Crohns disease, ulcerative colitis, chronic obstructive pulmonary disease, multiple sclerosis, atopic dermatitis, and graft versus host disease.
